SEQUENCE LISTING (132 sequences)

SEQ ID NO: 1 - protein, 1368 aa, Streptococcus pyogenes [sequence omitted]

SEQ ID NO: 2 - DNA, 4188 bp, Artificial Sequence
  synthetic
gtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatctatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataa

SEQ ID NO: 3 - protein, 8 aa, Artificial Sequence
  synthetic
APKKKRKV

SEQ ID NO: 4 - protein, 6 aa, Artificial Sequence
  synthetic
KKKKLK

SEQ ID NO: 5 - protein, 10 aa, Artificial Sequence
  synthetic
HHHHHHHHHH

SEQ ID NO: 6 - DNA, 607 bp, Bacillus subtilis
attcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaacta

SEQ ID NO: 7 - DNA, 247 bp, Artificial Sequence
  synthetic
acataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 8 - DNA, 50 bp, Artificial Sequence
  synthetic
atatatgagtaaacttggtctgacagaattcctccattttcttctgctat

SEQ ID NO: 9 - DNA, 35 bp, Artificial Sequence
  synthetic
tgcggccgcgaattcgattacgaatgccgtctccc

SEQ ID NO: 10 - DNA, 3290 bp, Artificial Sequence
  synthetic
gaattcgcggccgcacgcgtccatggggatccccgcgggtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgaca

SEQ ID NO: 11 - DNA, 4204 bp, Artificial Sequence
  synthetic
gcggccgcacgcgtccatggggatccccgcgggtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatggaattc

SEQ ID NO: 12 - DNA, 35 bp, Artificial Sequence
  synthetic
gggagacggcattcgtaatcgaattcgcggccgca

SEQ ID NO: 13 - DNA, 50 bp, Artificial Sequence
  synthetic
atagcagaagaaaatggaggaattctgtcagaccaagtttactcatatat

SEQ ID NO: 14 - DNA, 23 bp, Artificial Sequence
  synthetic
ccgactggagctcctatattacc

SEQ ID NO: 15 - DNA, 20 bp, Artificial Sequence
  synthetic
gctgtggcgatctgtattcc

SEQ ID NO: 16 - DNA, 22 bp, Artificial Sequence
  synthetic
gtcttttaagtaagtctactct

SEQ ID NO: 17 - DNA, 20 bp, Artificial Sequence
  synthetic
ccaaagcgattttaagcgcg

SEQ ID NO: 18 - DNA, 20 bp, Artificial Sequence
  synthetic
cctggcacgtggtaattctc

SEQ ID NO: 19 - DNA, 20 bp, Artificial Sequence
  synthetic
ggatttcctcaaatctgacg

SEQ ID NO: 20 - DNA, 21 bp, Artificial Sequence
  synthetic
gtagaaacgcgccaaattacg

SEQ ID NO: 21 - DNA, 20 bp, Artificial Sequence
  synthetic
gctggtggttgctaaagtcg

SEQ ID NO: 22 - DNA, 20 bp, Artificial Sequence
  synthetic
ggacgcaaccctcattcatc

SEQ ID NO: 23 - DNA, 20 bp, Artificial Sequence
  synthetic
caggcatccgatttgcaagg

SEQ ID NO: 24 - DNA, 19 bp, Artificial Sequence
  synthetic
gcaagcagcagattacgcg

SEQ ID NO: 25 - DNA, 8347 bp, Artificial Sequence
  synthetic
gaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatctatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatcgaattcgcggccgcacgcgtccatggggatccccgcgggtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgaca

SEQ ID NO: 26 - DNA, 9724 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggactcgacttcgaatacatccagttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacaaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttacacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcgaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatctatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 27 - DNA, 9724 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggagatgccatcagttcctcatagttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagagccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatctatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 28 - DNA, 20 bp, Bacillus licheniformis
ctcgacttcgaatacatcca

SEQ ID NO: 29 - DNA, 20 bp, Bacillus licheniformis
gatgccatcagttcctcata

SEQ ID NO: 30 - DNA, 1578 bp, Bacillus licheniformis
atgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaa

SEQ ID NO: 31 - DNA, 3 bp, Bacillus licheniformis
agg

SEQ ID NO: 32 - DNA, 20 bp, Bacillus licheniformis
ctcgacttcgaatacatcca

SEQ ID NO: 33 - DNA, 76 bp, Artificial Sequence
  synthetic
gttttagagctagaaatagcaagttaaaataaggctagtccgttatcaacttgaaaaagtggcaccgagtcggtgc

SEQ ID NO: 34 - RNA, 96 nt, Artificial Sequence
  synthetic
cucgacuucgaauacauccaguuuuagagcuagaaauagcaaguuaaaauaaggcuaguccguuaucaacuugaaaaaguggcaccgagucggugc

SEQ ID NO: 35 - DNA, 224 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtgga

SEQ ID NO: 36 - DNA, 95 bp, Artificial Sequence
  synthetic
gactcctgttgatagatccagtaatgacctcagaactccatctggatttgttcagaacgctcggttgccgccgggcgttttttattggtgagaat

SEQ ID NO: 37 - DNA, 500 bp, Bacillus licheniformis
aatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttac

SEQ ID NO: 38 - DNA, 40 bp, Artificial Sequence
  synthetic
tgagtaaacttggtctgacaaatggttctttcccctgtcc

SEQ ID NO: 39 - DNA, 46 bp, Artificial Sequence
  synthetic
aggttccgcagcttctgtgtaagatttcctcctaaataagcgtcat

SEQ ID NO: 40 - DNA, 500 bp, Bacillus licheniformis
acagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattc

SEQ ID NO: 41 - DNA, 46 bp, Artificial Sequence
  synthetic
atgacgcttatttaggaggaaatcttacacagaagctgcggaacct

SEQ ID NO: 42 - DNA, 41 bp, Artificial Sequence
  synthetic
cagaagaaaatggaggaattcgaatatcgaccggaacccac

SEQ ID NO: 43 - DNA, 415 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggactcgacttcgaatacatccagttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaat

SEQ ID NO: 44 - DNA, 1000 bp, Artificial Sequence
  synthetic
aatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttacacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattc

SEQ ID NO: 45 - DNA, 402 bp, Bacillus licheniformis
atgacgaactttggacaccatttacgacaattaagggaacggaaaaaactgaccgtcaatcaactggcgatgtattccggcgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactga

SEQ ID NO: 46 - DNA, 3 bp, Bacillus licheniformis
cgg

SEQ ID NO: 47 - DNA, 20 bp, Artificial Sequence
  synthetic
gatgccatcagttcctcata

SEQ ID NO: 48 - RNA, 96 nt, Artificial Sequence
  synthetic
gaugccaucaguuccucauaguuuuagagcuagaaauagcaaguuaaaauaaggcuaguccguuaucaacuugaaaaaguggcaccgagucggugc

SEQ ID NO: 49 - DNA, 500 bp, Bacillus licheniformis
ttgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggcc

SEQ ID NO: 50 - DNA, 40 bp, Artificial Sequence
  synthetic
tgagtaaacttggtctgacattgatattcagcaccctgcg

SEQ ID NO: 51 - DNA, 38 bp, Artificial Sequence
  synthetic
tgtgccgcggagaagtatggccaaaacctcgcaatctc

SEQ ID NO: 52 - DNA, 500 bp, Bacillus licheniformis
atacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagagccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaat

SEQ ID NO: 53 - DNA, 38 bp, Artificial Sequence
  synthetic
gagattgcgaggttttggccatacttctccgcggcaca

SEQ ID NO: 54 - DNA, 44 bp, Artificial Sequence
  synthetic
cagaagaaaatggaggaattcatttctcgggtttaaacagccac

SEQ ID NO: 55 - DNA, 415 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggagatgccatcagttcctcatagttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaat

SEQ ID NO: 56 - DNA, 1000 bp, Artificial Sequence
  synthetic
ttgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagagccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaat

SEQ ID NO: 57 - protein, 1368 aa, Artificial Sequence
  synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIHLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 58 - DNA, 33 bp, Artificial Sequence
  synthetic
gatctgcgtttaatccatcttgcgttagcgcac

SEQ ID NO: 59 - DNA, 33 bp, Artificial Sequence
  synthetic
gtgcgctaacgcaagatggattaaacgcagatc

SEQ ID NO: 60 - DNA, 9724 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggactcgacttcgaatacatccagttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacaaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttacacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcgaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatccatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 61 - DNA, 5042 bp, Artificial Sequence
  synthetic
attcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatccatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataaacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 62 - DNA, 9724 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggagatgccatcagttcctcatagttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagagccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatccatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 63 - DNA, 498 bp, Artificial Sequence
  synthetic
cacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatccatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttg

SEQ ID NO: 64 - DNA, 20 bp, Artificial Sequence
  synthetic
cacgtcgtaaaaatcgtatt

SEQ ID NO: 65 - DNA, 20 bp, Artificial Sequence
  synthetic
caaacagaccatttttcttt

SEQ ID NO: 66 - DNA, 8347 bp, Artificial Sequence; identical to SEQ ID NO: 25
  synthetic

SEQ ID NO: 67 - DNA, 7888 bp, Artificial Sequence
  synthetic
aaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatcgaattcgcggccgcacgcgtccatggggatccccgcgggtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtat

SEQ ID NO: 68 - DNA, 20 bp, Artificial Sequence
  synthetic
aaagaaaaatggtctgtttg

SEQ ID NO: 69 - DNA, 20 bp, Artificial Sequence
  synthetic
aatacgatttttacgacgtg

SEQ ID NO: 70 - DNA, 9790 bp, Artificial Sequence
  synthetic
gaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatccatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatcgggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaagaatcgaaaacggccaccggttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgctaagttcccaagcccgcgacgatcagaaaattggcctgataactgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgcgaattcgcggccgcacgcgtccatggggatccccgcgggtcgacctcgagagttacgctagggataacagggtaatataggagctccagtcggcttaaaccagttttcgctggtgcgaaaaaagagtgtcttgtgacactcttaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgtaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtcgggaagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgacccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgaca

SEQ ID NO: 71 - DNA, 399 bp, Bacillus licheniformis
atgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataa

SEQ ID NO: 72 - DNA, 1438 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaagaatcgaaaacggccaccggttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgctaagttcccaagcccgcgacgatcagaaaattggcctgataactgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgc

SEQ ID NO: 73 - DNA, 1023 bp, Artificial Sequence
  synthetic
cgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgctaagttcccaagcccgcgacgatcagaaaattggcctgataactgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgc

SEQ ID NO: 74 - DNA, 415 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaagaatcgaaaacggccaccggttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaat

SEQ ID NO: 75 - DNA, 17 bp, Artificial Sequence
  synthetic
cgtgcggccgcgaattc

SEQ ID NO: 76 - DNA, 29 bp, Artificial Sequence
  synthetic
cctgataccgggagacggcattcgtaatc

SEQ ID NO: 77 - DNA, 8352 bp, Artificial Sequence
  synthetic
gaattcgcggccgcacgcgtccatggggatccccgcgggtcgacctcgagagttacgctagggataacagggtaatataggagctccagtcggcttaaaccagttttcgctggtgcgaaaaaagagtgtcttgtgacactcttaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgtaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtcgggaagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgacccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatccatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 78 - DNA, 17 bp, Artificial Sequence
  synthetic
gaattcgcggccgcacg

SEQ ID NO: 79 - DNA, 29 bp, Artificial Sequence
  synhtetic
gattacgaatgccgtctcccggtatcagg

SEQ ID NO: 80 - DNA, 9706 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggagatgccatcagttcctcatagttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatccatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 81 - DNA, 23 bp, Artificial Sequence
  synthetic
gatgccatcagttcctcatacgg

SEQ ID NO: 82 - DNA, 982 bp, Artificial Sequence
  synthetic
ttgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaat

SEQ ID NO: 83 - DNA, 9738 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggagcgagcggctcaaagagctggttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacacgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggcaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacgggaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatccatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 84 - DNA, 23 bp, Artificial Sequence
  synhtetic
gcgagcggctcaaagagctgagg

SEQ ID NO: 85 - DNA, 1014 bp, Artificial Sequence
  synthetic
cgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggcaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacgg

SEQ ID NO: 86 - DNA, 9744 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggagatgtattccggcgtcagttgttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatgacgaaccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatccatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 87 - DNA, 23 bp, Artificial Sequence
  synthetic
gatgtattccggcgtcagttcgg

SEQ ID NO: 88 - DNA, 1020 bp, Artificial Sequence
  synthetic
gatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatgacgaaccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacg

SEQ ID NO: 89 - DNA, 9732 bp, Artificial Sequence
  synthetic
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggagatgtattccggcgtcagttgttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacaccacgtttagtcttgaaacaaactttatcaatcggccggccccttcagacaaagaccggcaaataggaaaaaggcctgacttgaacatcagacctcatgcttcattgtcttatacaaagtaagcaagcgcaatcgttaagaaaaagaaaagcacggttaaaacgaccgtcatccggtgaagaatcaaatcaagtcctcttgctttctgctttccgaaaagctgctcggctccgccggaaatcgcgcctgataatccggcgcttttgccggattgcagtaaaacgacaacgattaatacgatacttacaatcactaataagaccgttaaaaatgcggccgtaacctacacctccagacaaactggctgacaatagttttattttacatgaaaagcaagcgcatgtcacgagcgtttcgaacagctttttttattttttcccagcgccggaataaggtatacaaaaaaagagcggctctgctccctttcctgcggaatatgtaatcacataaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatccatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 90 - DNA, 1008 bp, Artificial Sequence
  synthetic
ccacgtttagtcttgaaacaaactttatcaatcggccggccccttcagacaaagaccggcaaataggaaaaaggcctgacttgaacatcagacctcatgcttcattgtcttatacaaagtaagcaagcgcaatcgttaagaaaaagaaaagcacggttaaaacgaccgtcatccggtgaagaatcaaatcaagtcctcttgctttctgctttccgaaaagctgctcggctccgccggaaatcgcgcctgataatccggcgcttttgccggattgcagtaaaacgacaacgattaatacgatacttacaatcactaataagaccgttaaaaatgcggccgtaacctacacctccagacaaactggctgacaatagttttattttacatgaaaagcaagcgcatgtcacgagcgtttcgaacagctttttttattttttcccagcgccggaataaggtatacaaaaaaagagcggctctgctccctttcctgcggaatatgtaatcacataaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaa

SEQ ID NO: 91 - DNA, 2793 bp, Bacillus licheniformis
ttatacaaagtaagcaagcgcaatcgttaagaaaaagaaaagcacggttaaaacgaccgtcatccggtgaagaatcaaatcaagtcctcttgctttctgctttccgaaaagctgctcggctccgccggaaatcgcgcctgataatccggcgcttttgccggattgcagtaaaacgacaacgattaatacgatacttacaatcactaataagaccgttaaaaatgcggccataacctacacctccagacaaactggctgacaatagttttattttacatgaaaagcaagcgcatgtcacgagcgtttcgaacagctttttttattttttcccagcgccggaataaggtatacaaaaaaagagcggctctgctccctttcctgcggaatatgtaatcacatttatttcttttctgacagtgccgccatcatatcttccaggagcatttccgctccgcgcgggctgagtacgattttgccgcccgcatacgttttatttttcgtggtgatgtcgccggtcatcaaacatgccatgtttgcggcgtattttgtcaaaatgatattttcgccgtcggtatatatttcgagcgggtccttttcattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatgacgaactttggacaccatttacgacaattaagggaacggaaaaaactgaccgtcaatcaactggcgatgtattccggcgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgccgacattaaaaagtggcggtaaaattcccccagcacatagccgccgttttgcgcgtaatacgtccaaagctgctgcagcacttccat

SEQ ID NO: 92 - DNA, 1320 bp, Bacillus licheniformis
ttgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgatacagcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataa

SEQ ID NO: 93 - DNA, 7237 bp, Artificial Sequence
  synthetic
tctgaccaaagactcctgcttcaatcgttgaacgctggctgccacctgctctctcgacagagccgaacaaaagccgaagtattttgaaacggcaaataaaccggcgtcctgtatcgtctgtgacgacctttttccttttaataaatgatagaccgcgcttggagaacgctcacccttcatggatgacagaatgtcaagcacaatcgcgtcaaaaaaatgaaccggcatatcatcacctgcaatcttccggcaacattcgatcatttcttccttttattttaacagattttgcggagaaatcgacgtttaaactcatataaaaggggtatgttagcagtagaacccttgtgtgataagcattctcaatatttttgagttgaaatgtaagattaacaccattacaataaggaatgggaataggtttcatatcggatagatagagggttaaaccatttgttccaacgaagaacaatctgggaggttttttattcatgccaaaatatacaattgtagacaaagatacgtgcatcgcatgcggagcttgtggtgctgcggctcctgatatttatgattacgacgatgagggaatcgcatttgtcacccttgacgacaatcagggtgtcgtcgaagtccctgacgtcttagaagaagacatgatggacgcgtttgaaggctgtcctacagattcgatcaaagttgcggatgagccgttcgaaggcgacccgcttaaacacgaataaagccaaaaaacatccggtgcacaaagtgccggatgtttttttatgagataagcacggctttaccaacaagcaaaaagaagccggctaaagacatccggcttcttctgcagctgacaatatccgggaacatgcacccgatattgtcatgtttatttatttggccatgcggacgttttccttcagccgcggtttcagcgaaaggaaaatcggcgtggacacgagggccacagcgatgcctttaatgaaattaaaaggcaggattccggccagaactgttgtcttgagcgcctctccagtcagcgctggagcatttaaaaaccaagtgtaggcaggcagaaacagcagataatttaaaatgctcatcgaaacggccatcacaagcgtccctgcgaaaagagctgtgacaaaccctttggcagaacttgattttttcagcagtacagctgccggcaggataaacaatgttccggcaatgaagttagccgcctgatcaatcggaacgcccgaggcgcttcctgcaataaagtaattcagcacgtttttgatcgcttcaacggcaatcccggctcccggaccgtacaaaataacagcgagcaatgccgggatatcactgaaatcgatttttaaatacgggaatgcccccaggatcggaaagctcagcatcattaaaataaatgcgatgctgctcagcatgctgatagagacgagacgtctcaccttgttgtgtttcattttgtcactctctccttttcgatcacatctcacgaaaagaggaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgagctctcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagaatgaaacaacaaaaacggctttacgcccgattgctgacgctgttatttgcgctcatcttcttgctgcctcattctgcagctagcgcagcagcgacaaacggaacaatgatgcagtatttcgagtggtatgtacctaacgacggccagcaatggaacagactgagaacagatgccccttacttgtcatctgttggtattaacgcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaacacgctgcattcaaatggaatccaagtgtatggtgatgtcgtgatgaatcataaagcaggtgctgattatacagaaaacgtaacggcggtggaggtgaatccgtctaatagatatcaggaaatcagcggcgaatataatattcaggcatggacaggcttcaactttccgggcagaggaacaacgtattctaactggaaatggcagtggttccattttgatggaacggattgggaccagagcagaagcctctctagaatcttcaaattcgatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggatattacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatccttctgtattttatggagatatgtacggtacaaaaggaacgacaacaagagagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaataaggaatcagtttcagtatgggtgcagcaatgaaagcttctcgaggttaacagaggacggatttcctgaaggaaatccgtttttttattttgcggccgcatattccgcattcgcaatgcctaccgcatactaaaaaccgcacattcacagttatttcatttttaattttcgtctttccgcgtgaaactcattgacactctttatggaatatggtaaattatcagatatttatgacgcttatttaggaggaaatcttacatgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaaacagaagctgcggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcccgtattgccgatgattccgatttgctcgccttttttcacccgctccttttcctttttcaggcgtttgcttaagtgggcataaacggtttcatatccgttgtcatgtttaataaatatcacttggccgtaggagtcggattgatacgatttgcttatcgttccgtctgcggctgccgctactgcttccccttcgggagcagcgatgtcaagccccttatgctttccgcctctcgtaccgaattgatctgtgatctctcctttaatcggttcaatccactctgaggcttccgcccccggggcattgacgaaaagcgccaatcccgaaagccatgcgatcgcgaacaggaagttttgatgtctgagtttcttcaaggttttccatatcctcctattacatgcatcttcggtaaaattgccccctattcggagacagcttagtatacttccaaatcaatacaatttatacattaaaaaaagactccgcacagggagtcttttagttttctatcgtcatcggattcggtgcgtacggaacctgtacagatttcgacaggtcataggcgccgaccttggttatggatgcgtttttaaatttcacttttgtgaagccgaaatctttcgcggtcaatagaaggccttccaccatcaagacatcttcgggtttattttcaatattcgcggaggaagaaaattgaatgatcagttcttttccattcttttgaatatcttcaatcggcgtatcatcggataaaatgggttttaaatgagtgccgctttcttcgtttttcatcatcttaatcgcttcctgcaccgattcgtaagattcgcttgaaggtgcaaggaaccggcgcccgtctgagctttcatataaatagtagcatttttgcgtctggtgcataatcgccatatcggcgagcattccgaatgtttcaaattcaacacccgatttatcattggaaataaacagaacagaatcatacgatccccatttaaaggtttcgttgatcacatttttcagccgttcgaaatcttcgactgatagctccggtattttctcatcaacttgaatcttcagttttttattgtttttctgctctttgaacttcaccttatcaaggtaagctgtgtcaaatgatgtaaactggtccactccaagccggctgtaagcgtgaagcgcatcttcaagatttgtcatgccagtgcttttctcgaggcttaccgggacaacgacagacttggactcgtcaaggaaagcgaaggtgatatagtcgtctttttgattctgtgagacgacaaacgtatttgcaggttcagacttggcagcatcagcctccgtctgcaccaattttccgtcag

SEQ ID NO: 94 - DNA, 94 bp, Artificial Sequence
  synthetic
tcgctgataaacagctgacatcaatatcctattttttcaaaaaatattttaaaaagttgttgacttaaaagaagctaaatgttatagtaataaa

SEQ ID NO: 95 - DNA, 58 bp, Bacillus subtilis
acagaatagtcttttaagtaagtctactctgaatttttttaaaaggagagggtaaaga

SEQ ID NO: 96 - DNA, 87 bp, Bacillus licheniformis
atgaaacaacaaaaacggctttacgcccgattgctgacgctgttatttgcgctcatcttcttgctgcctcattctgcagctagcgca

SEQ ID NO: 97 - DNA, 1452 bp, Artificial Sequence
  synthetic
gcagcgacaaacggaacaatgatgcagtatttcgagtggtatgtacctaacgacggccagcaatggaacagactgagaacagatgccccttacttgtcatctgttggtattaacgcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaacacgctgcattcaaatggaatccaagtgtatggtgatgtcgtgatgaatcataaagcaggtgctgattatacagaaaacgtaacggcggtggaggtgaatccgtctaatagatatcaggaaatcagcggcgaatataatattcaggcatggacaggcttcaactttccgggcagaggaacaacgtattctaactggaaatggcagtggttccattttgatggaacggattgggaccagagcagaagcctctctagaatcttcaaattcgatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggatattacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatccttctgtattttatggagatatgtacggtacaaaaggaacgacaacaagagagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaataaggaatcagtttcagtatgggtgcagcaatga

SEQ ID NO: 98 - DNA, 34 bp, Bacillus licheniformis
cggatttcctgaaggaaatccgtttttttatttt

SEQ ID NO: 99 - DNA, 7487 bp, Artificial Sequence
  synthetic
caaaatagaaaagccgcggttcacacggagtattacaaagacatcagttcgctttcttttccggtattcagcgatttgaaggaagaggatgccaagctggccaacgatgcggtaaaacttcatttaaaaaattcctataaagaatttcaaaaaatcgttaatgatgccgaaaagaaggataaggatgaagaaaacgtttatgaaacgtcctacaaagtcaaatacaacgaggaaggcaaactgagctttttaatctatgactatcagttctccggcggtgcgcacggcatgtacaccgtaacatcctacaactttgactttgacaagcataaacaagtcgtgctgactgacgtattaaacaatcaggcgaaaatcgaaaaggcaaaaaactatattttcagctatatcaacgaacatccggaacagttttattctgatcttaaaaagagcgatatccgtttggatgaacatacggcattctattatacaagcagcggaatttcaattgtatttcagcagtatgatatcgccccgtatgcagccggaaaccaggaaataaagcttccgtcgacgcttttatattagccccggcattagatctaatatttgtaatagaaacagagagagcaagtcgtgaaacaggagagtgagcagcgatgtctggcaaaccatcatttcgatgggttaaaatgttgatttttttaacgatattaataggtttggcagggtactcttacaataaagtgtcaagcaacagccaagagccccctcagccaaaaaaagaccgcggacaatccggcctcggcgtcgaatccatggtcaatgacagcaaacaagagaggtatgccatccattatccggtgtttcacataaaagaaatcgatgaacaaataaaagattatgtgaatcaagaattggccggttttaaagaggataacgcaaaggcccaggctcaggatgaagacgggccttttgaactgaacattaaatataaggttgtctattatacaaaggatacggccagtgttgtgctgaatcaatacatagaggccggcggcgtatcgggtacaacatctgtcaagacgtttaacgctgatttaaagcagaaaaagctgctgtcccttcaagatctgtttgaagagaattcagattttctgaacaggatttcaagcattgcctatcaggaattgaaaaatcggaatccgtctgctgacatggcttttttaaaagaagggacgagccctcaggaagaacatttcagccgctttgcgcttcttgaaaacgaggtggaattttattttgagaaaaaacaagccggtcttgaacagtttgtaaaaataaaaaaagaatgggtaaaagatattttaaaagaccgatatcaggatatgaaaaagaatcgtcttcaggccaaacctgatcaggagcctgttccgcttccgaagcaagcgaaaattaatcccgatgaaaaagtgattgccctcacatttgatgacggtccgaatcccgctacaacgaataaaatattaaacgctttacagaagcatgaagggcatgcgaccttctttgtgcttggaagcagagcccaatattatcccgaaacgataaaacggatgctgaaggaaggaaacgaagtcggcaaccattcctgggaccatccgttattgacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggcgcgaaaatctcagcttttcggctctttttttattgaatggacgttgtgtatgcctatttctatcaagcgctgttttctgttattctataatcaatagaatggattagttgtttagggaatcatttcctttataaatcaagaaaatttggacaaatggtggtttagtttttaaaacgaaatgttataatacaacataagaatcgcactatcatgaagccggaagatgcatcgggcagcaaccggagcgccccttgcacctttgtcgatagagaaagagggaatgacaattgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgatacagcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataaaaaaatggagattccctaagaggggggtctccatttttaattcagcttttcttttggaagaaaatatagggaaaatggtacttgttaaaaattcggaatatttatacaatatcatatgacagaatagtcttttaagtaagtctactctgaatttttttaaaaggagagggtaaagaatgaaacaacaaaaacggctttacgcccgattgctgacgctgttatttgcgctcatcttcttgctgcctcattctgcagctagcgcagcagcgacaaacggaacaatgatgcagtatttcgagtggtatgtacctaacgacggccagcaatggaacagactgagaacagatgccccttacttgtcatctgttggtattaacgcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaacacgctgcattcaaatggaatccaagtgtatggtgatgtcgtgatgaatcataaagcaggtgctgattatacagaaaacgtaacggcggtggaggtgaatccgtctaatagatatcaggaaatcagcggcgaatataatattcaggcatggacaggcttcaactttccgggcagaggaacaacgtattctaactggaaatggcagtggttccattttgatggaacggattgggaccagagcagaagcctctctagaatcttcaaattcgatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggatattacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatccttctgtattttatggagatatgtacggtacaaaaggaacgacaacaagagagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaataaggaatcagtttcagtatgggtgcagcaatgaaagagcagagaggacggatttcctgaaggaaatccgtttttttattttgcccgtcttataaatttctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattattgcgacggggttgagaaaagccgattttgaagggctcatcaaggtttttgaaaagcctgtcgttgtattcgggcaaaatgaaatgggctacgattttattgatgttaacaatgaaaaaggaacctatatggcaacacgtcacgtcattggtctgggcgtccgcaatgtcgtcttttttgggatcgatttggatgagccctttgaacgctcaagggaaaaaggctatcttcaggcgatggaaggcagtctgaaaaaagcagcgattttccggatggaaaacagttcaaaaaaaagtgaagcacgcgcgcgggaagtgcttgcatcctttgacgcacctgcagcggttgtttgcgcttcggaccgaatcgcgctcggggttatccgcgcggtgcaatcgcttggtaaaagaattccggaagatgtcgcggtcaccggctatgacggggtgtttctcgaccggatcgcttcgcctcgcctgacaaccgtcagacagcctgttgttgaaatgggagaggcttgcgcgagaatcctgctgaaaaaaatcaatgaagacggagcgccgcaaggcaatcaattttttgagccggagcttattgtccgcgaatcgactttgtagggtgtctcattctgttaccgttaacaagctgaaaatgattgttcctgttaccgccgtcatgataatttcagaataaaagccggtttatcacagccggacaaccaaaagggggaaacatgatggaatatgcagcgatacatcatcagcctttcagctctgatgcctattcttacaatggacggacattgcacatcaagatccgtacaaaaaaggatgatgccgaacacgtccgcttggtttggggcgatccttacgaatacaccggcggcacatggaaagcgaacgagcttgcgatggcgaaaattgccgcaacaagcacccatgattactggtttgccgaagtggcgcctccattcaggcgtctgcaatacggatttatcctgacaggcgctgatgatcgagacactttttacggaagcaatggtgcatgtccgtttgccgggaaagcggcggatataggcaaacactgttttaaatttccgtttgttcatgaggcagacacgtttgatgcacctgactgggtcaaatcaaccgtctggtatcaaatttttccggagcgctttgccagcgggcgggaagatttgtctccggaaaacgctttgccatggggaagcaaagatcctgaggcgcacgattttttcggaggggatttgcaggggatcatggacaagctggactatttggaagacttgggggtaggcggaatctatttgacgccgatctttgccgcgccttccaaccataaatacgacacattggactattgctccatcgatccgcattttggcgatgaggagctctttcgcacgctggtcagccggattcacgagcggggaatgaaaatcatgcttgatgctgtttttaaccacattggcagcgcttcgcaagagtggcaggatgttgtcaaaaacggtgaaacgtcccgctataaagactggttccatattcattctttccctgttaaagaaggcagctatgatacatttgc

SEQ ID NO: 100 - DNA, 74 bp, Bacillus licheniformis
gcttttcttttggaagaaaatatagggaaaatggtacttgttaaaaattcggaatatttatacaatatcatatg

SEQ ID NO: 101 - DNA, 6393 bp, Artificial Sequence
  synthetic
aagcttcatatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatggatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgggcgcgccggtatccgcgcttcttgagcactatttattcaaagccgctccagatcaatagcgctttttcagctccctgaggatgaattcgtatatcagctgattccggtcttctttcggatagagcataaattcctgtttcttctgcatggggtttccttcaatcctgtcgataaattttgttctcagccatgccgttcggtaaacctggttttcgaaagatgagatggatacgggcagctccagcgtttccccgttgacaaacgtgacaaacgtgttgtcatactttgccgcgcaaaactcgtgaacatgcgcatgggaaagccacccgcactgaggacgagttgaggaaaatgtggggaaaagaaaaatgttgtttgagtgatccaccatgatcggcggtttatgggaaactttaatgacttcatatgtgcccgcttttcttcccgcatagctcgatccgaaatagcggcagcttctttcgataatttgaaacggcttcatattgacgcggaaagtcctgtcggtctcaagtatttttgaggcggatttctccccctcacccagaggcaggacagccattgtcgaactgtttacttcatacgtatcctttgtcatatcctctgtgctcatgtgatttcccccttaaaaataaattcattcaaatacagatgcattttatttcatatagtaagtacatcacctattagtttgttgtttaaacaaactaacttattttcatcttatataacctcgtcagtattttcaatattttttttagttttttatgaacacattagatttaataaagggaagattcgctatgtactatgttgatacttaatttaaagattaaacaaatggagtggatgaagtggatatcgctgatcaaacctttgtcaaaaaagtaaatcaaaagttattattaaaagaaatccttaaaaattcacctatttcaagagcaaaattatctgaaatgactggattaaataaatcaactgtctcatcacaggtaaacacgttaatgaaagaaagtatggtatttgaaataggtcaaggacaatcaagtggcggaagaagacctgtcatgcttgtttttaataaaaaggcaggatactccgttggaatagatgttggtgtggattatattaatggcattttaacagaccttgaaggaacaatcgttcttgatcaataccgccatttggaatccaattctccagaaataacgaaagacattttgattgatatgattcatcactttattacgcaaatgccccaatctccgtacgggtttattggtataggtacttgcgtgcctggactcattgataaagatcaaaaaattgttttcactccgaactccaactggagagatattgacttaaaatcttcgatacaagagaagtacaatgtgtctgtttttattgaaaatgaggcaaatgctggcgcatatggagaaaaactatttggagctgcaaaaaatcacgataacattatttacgtaagtatcagcacaggaatagggatcggtgttattatcaacaatcatttatatagaggagtaagcggcttctctggagaaatgggacatatgacaatagactttaatggtcctaaatgcagttgcggaaaccgaggatgctgggaattgtatgcttcagagaaggctttatttaaatctcttcagaccaaagagaaaaaactgtcctatcaagatatcataaacctcgcccatctgaatgatatcggaaccttaaatgcattacaaaattttggattctatttaggaataggccttaccaatattctaaatactctcaacccacaagccgtaattttaagaaatagcataattgaatcgcatcctatggttttaaattcaatgagaagtgaagtatcatcaagggtttattcccaattaggcaatagctatgaattattgccatcttccttaggacagaatgcaccggcattaggaatgtcctccattgtgattgatcattttctggacatgattacaatgtaattttttatggaatggacagctcatctttaaagatgagtttttttattctaggagtatttctgaagcaatagtgacatggcaccttctcatatgaaaaaggagttctaaaataaaaatctcctttttcatgtgcaaattatttttctttataacgaaaatatctaaatgacaatgcatatgcaagaggggatcacataaatatatattttaaaaatatcccactttatccaattttcgtttgttgaactaatgggtgctttagttgaagaataaaagaccacattaaaaaatgtggtcttttgtgtttttttaaaggatttgagcgtagcgaaaaatccttttctttcttatcttgatactatatagaaacaacatcatttttcaaaattaggtcaaagccttgtgtatcaagggtttgatggttctttgacaggtaaaaactccttctgctattattaaatactatatagaaacaacatcatttttcaaaattaggtcaaagccttgtgtatcaagggtttgatggttctttgacaggtaaaaactccttctgctattattaaggtgtcgaatcaaaataatagaatgctagagaactagctcagaaggagtttttttgttgatttattcatctgaaaatgattatagcatcctcgaagataaaaccgcaacaggtaaaaagcgggattggaaggggaaaaagagacggacgaacctcatggcggagcattacgaagcgttagagagtaagattggggcaccttactatggcaaaaaggctgaaaaactaattagttgtgcagagtatctttcgtttaagagagacccggagacgggcaagttaaaactgtatcaagcccatttttgtaaagtgaggttatgtccgatgtgtgcgtggcgcaggtcgttaaaaattgcttatcacaataagttgatcgtagaggaagccaatagacagtacggctgcggatggatttttctcacgctgacgattcgaaatgtaaagggagaacggctgaagccacaaatttctgcgatgatggaaggctttaggaaactgttccagtacaaaaaagtaaaaacttcggttcttggatttttcagagctttagagattaccaaaaatcatgaagaagatacatatcatcctcattttcatgtgttgataccagtaaggaaaaattattttgggaaaaactatattaagcaggcggagtggacgagcctttggaaaaaggcgatgaaattggattacactccaattgtcgatattcgtcgagtgaaaggtaaagctaagattgacgctgaacagattgaaaacgatgtgcggaacgcaatgatggagcaaaaagctgttctcgaaatctctaaatatccggttaaggatacggatgttgtgcgcggtaataaggtgactgaagacaatctgaacacggtgctttacttggatgatgcgttggcagctcgaaggttaattggatacggtggcattttgaaggagatacataaagagctgaatcttggtgatgcggaggacggcgatctggtcaagattgaggaagaagatgacgaggttgcaaatggtgcatttgaggttatggcttattggcatcctggcattaaaaattacataatcaaataaaaaaagcagacctttagaaggcctgcttttttaactaacccatttgtattgtgttgaaatatgttttgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagcgattcacaaaaaataggcacacgaaaaacaagttaagggatgcagtttatgcatcccttaacttaaaatactaaaaatgcccatattttttcctccttataaaattagtataattatagcacgagatctaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccggatcc

SEQ ID NO: 102 - DNA, 765 bp, Artificial Sequence
  synthetic
gtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaatta

SEQ ID NO: 103 - DNA, 1161 bp, Bacillus licheniformis
gtggatgaagtggatatcgctgatcaaacctttgtcaaaaaagtaaatcaaaagttattattaaaagaaatccttaaaaattcacctatttcaagagcaaaattatctgaaatgactggattaaataaatcaactgtctcatcacaggtaaacacgttaatgaaagaaagtatggtatttgaaataggtcaaggacaatcaagtggcggaagaagacctgtcatgcttgtttttaataaaaaggcaggatactccgttggaatagatgttggtgtggattatattaatggcattttaacagaccttgaaggaacaatcgttcttgatcaataccgccatttggaatccaattctccagaaataacgaaagacattttgattgatatgattcatcactttattacgcaaatgccccaatctccgtacgggtttattggtataggtacttgcgtgcctggactcattgataaagatcaaaaaattgttttcactccgaactccaactggagagatattgacttaaaatcttcgatacaagagaagtacaatgtgtctgtttttattgaaaatgaggcaaatgctggcgcatatggagaaaaactatttggagctgcaaaaaatcacgataacattatttacgtaagtatcagcacaggaatagggatcggtgttattatcaacaatcatttatatagaggagtaagcggcttctctggagaaatgggacatatgacaatagactttaatggtcctaaatgcagttgcggaaaccgaggatgctgggaattgtatgcttcagagaaggctttatttaaatctcttcagaccaaagagaaaaaactgtcctatcaagatatcataaacctcgcccatctgaatgatatcggaaccttaaatgcattacaaaattttggattctatttaggaataggccttaccaatattctaaatactctcaacccacaagccgtaattttaagaaatagcataattgaatcgcatcctatggttttaaattcaatgagaagtgaagtatcatcaagggtttattcccaattaggcaatagctatgaattattgccatcttccttaggacagaatgcaccggcattaggaatgtcctccattgtgattgatcattttctggacatgattacaatg

SEQ ID NO: 104 - DNA, 66 bp, Bacillus licheniformis
tgtacttactatatgaaataaaatgcatctgtatttgaatgaatttatttttaagggggaaatcac

SEQ ID NO: 105 - DNA, 576 bp, Bacillus licheniformis
atgagcacagaggatatgacaaaggatacgtatgaagtaaacagttcgacaatggctgtcctgcctctgggtgagggggagaaatccgcctcaaaaatacttgagaccgacaggactttccgcgtcaatatgaagccgtttcaaattatcgaaagaagctgccgctatttcggatcgagctatgcgggaagaaaagcgggcacatatgaagtcattaaagtttcccataaaccgccgatcatggtggatcactcaaacaacatttttcttttccccacattttcctcaactcgtcctcagtgcgggtggctttcccatgcgcatgttcacgagttttgcgcggcaaagtatgacaacacgtttgtcacgtttgtcaacggggaaacgctggagctgcccgtatccatctcatctttcgaaaaccaggtttaccgaacggcatggctgagaacaaaatttatcgacaggattgaaggaaaccccatgcagaagaaacaggaatttatgctctatccgaaagaagaccggaatcagctgatatacgaattcatcctcagggagctgaaaaagcgctat

SEQ ID NO: 106 - DNA, 20 bp, Artificial Sequence
  synthetic
gcgaatcgaaaacggaaagc

SEQ ID NO: 107 - DNA, 20 bp, Artificial Sequence
  synthetic
tcatcgcgatcggcattacg

SEQ ID NO: 108 - DNA, 1146 bp, Bacillus licheniformis
gcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatga

SEQ ID NO: 109 - DNA, 1146 bp, Artificial Sequence
  synthetic
gcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgctaagttcccaagcccgcgacgatcagaaaattggcctgataactgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatga

SEQ ID NO: 110 - DNA, 20 bp, Artificial Sequence
  synthetic
tttcgactttctcgtgcagg

SEQ ID NO: 111 - DNA, 20 bp, Artificial Sequence
  synthetic
atcaaacatgccatgtttgc

SEQ ID NO: 112 - DNA, 18 bp, Artificial Sequence
  synthetic
aggttgagcaggtcttcg

SEQ ID NO: 113 - DNA, 1499 bp, Bacillus licheniformis
atcaaacatgccatgtttgcggcgtattttgtcaaaatgatattttcgccgtcggtatatatttcgagcgggtccttttcattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatgacgaactttggacaccatttacgacaattaagggaacggaaaaaactgaccgtcaatcaactggcgatgtattccggcgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacct

SEQ ID NO: 114 - DNA, 1097 bp, Artificial Sequence
  synthetic
atcaaacatgccatgtttgcggcgtattttgtcaaaatgatattttcgccgtcggtatatatttcgagcgggtccttttcattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacct

SEQ ID NO: 115 - DNA, 20 bp, Artificial Sequence
  synthetic
gagattgcgaggttttggcc

SEQ ID NO: 116 - DNA, 20 bp, Artificial Sequence
  synthetic
ggcatacggcgtattgttcg

SEQ ID NO: 117 - DNA, 1629 bp, Bacillus licheniformis
gagattgcgaggttttggccatgacgaactttggacaccatttacgacaattaagggaacggaaaaaactgaccgtcaatcaactggcgatgtattccggcgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgcc

SEQ ID NO: 118 - DNA, 1248 bp, Artificial Sequence
  synthetic
gagattgcgaggttttggccatgacgaactttggacaccatttacgacaattaagggaacggaaaaaactgaccgtcaatcaactggcgatgtattccggcgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggcaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgcc

SEQ ID NO: 119 - DNA, 20 bp, Artificial Sequence
  synthetic
atgatattttcgccgtcggt

SEQ ID NO: 120 - DNA, 20 bp, Artificial Sequence
  synthetic
aacgatgcaggagctcaatt

SEQ ID NO: 121 - DNA, 2353 bp, Bacillus licheniformis
atgatattttcgccgtcggtatatatttcgagcgggtccttttcattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatgacgaactttggacaccatttacgacaattaagggaacggaaaaaactgaccgtcaatcaactggcgatgtattccggcgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgccgacattaaaaagtggcggtaaaattcccccagcacatagccgccgttttgcgcgtaatacgtccaaagctgctgcagcacttccatttgtcatcccctccttttactcaaaataattgtgcttctttaaaaactcggccgcgacgacagaaggctctttcagctttccatcgacttcgtaattgagctcctgcatcgtt

SEQ ID NO: 122 - DNA, 1401 bp, Artificial Sequence
  synthetic
atgatattttcgccgtcggtatatatttcgagcgggtccttttcattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatgacgaaccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgccgacattaaaaagtggcggtaaaattcccccagcacatagccgccgttttgcgcgtaatacgtccaaagctgctgcagcacttccatttgtcatcccctccttttactcaaaataattgtgcttctttaaaaactcggccgcgacgacagaaggctctttcagctttccatcgacttcgtaattgagctcctgcatcgtt

SEQ ID NO: 123 - DNA, 20 bp, Artificial Sequence
  synthetic
catgacgtctttccaccagt

SEQ ID NO: 124 - DNA, 20 bp, Artificial Sequence
  synthetic
aacgatgcaggagctcaatt

SEQ ID NO: 125 - DNA, 3265 bp, Bacillus licheniformis
catgacgtctttccaccagtcttccggtcctgtggagagaagttcttctggaggcacgccgtggcctttatattggggcgcatggcatgtgtagcctttttcattcaaatatcttccgagcattctaacgtccgccgtgttgcccgtaaatccgtgcagcagcaggacggctttttttccgcctttaaatgtgaaaggttgtggtttgacaattttcatgattcactgtctccttttcatatgtattcttccacgtttagtcttgaaacaaactttatcaatcggccggccccttcagacaaagaccggcaaataggaaaaaggcctgacttgaacatcagacctcatgcttcattgtcttatacaaagtaagcaagcgcaatcgttaagaaaaagaaaagcacggttaaaacgaccgtcatccggtgaagaatcaaatcaagtcctcttgctttctgctttccgaaaagctgctcggctccgccggaaatcgcgcctgataatccggcgcttttgccggattgcagtaaaacgacaacgattaatacgatacttacaatcactaataagaccgttaaaaatgcggccataacctacacctccagacaaactggctgacaatagttttattttacatgaaaagcaagcgcatgtcacgagcgtttcgaacagctttttttattttttcccagcgccggaataaggtatacaaaaaaagagcggctctgctccctttcctgcggaatatgtaatcacatttatttcttttctgacagtgccgccatcatatcttccaggagcatttccgctccgcgcgggctgagtacgattttgccgcccgcatacgttttatttttcgtggtgatgtcgccggtcatcaaacatgccatgtttgcggcgtattttgtcaaaatgatattttcgccgtcggtatatatttcgagcgggtccttttcattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatgacgaactttggacaccatttacgacaattaagggaacggaaaaaactgaccgtcaatcaactggcgatgtattccggcgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgccgacattaaaaagtggcggtaaaattcccccagcacatagccgccgttttgcgcgtaatacgtccaaagctgctgcagcacttccatttgtcatcccctccttttactcaaaataattgtgcttctttaaaaactcggccgcgacgacagaaggctctttcagctttccatcgacttcgtaattgagctcctgcatcgtt

SEQ ID NO: 126 - DNA, 3265 bp, Artificial Sequence; identical to SEQ ID NO: 125
  synthetic

SEQ ID NO: 127 - DNA, 2793 bp, Bacillus licheniformis; identical to SEQ ID NO: 91

SEQ ID NO: 128 - DNA, 2793 bp, Artificial Sequence
  synthetic
ttatacaaagtaagcaagcgcaatcgttaagaaaaagaaaagcacggttaaaacgaccgtcatccggtgaagaatcaaatcaagtcctcttgctttctgctttccgaaaagctgctcggctccgccggaaatcgcgcctgataatccggcgcttttgccggattgcagtaaaacgacaacgattaatacgatacttacaatcactaataagaccgttaaaaatgcggccataacctacacctccagacaaactggctgacaatagttttattttacatgaaaagcaagcgcatgtcacgagcgtttcgaacagctttttttattttttcccagcgccggaataaggtatacaaaaaaagagcggctctgctccctttcctgcggaatatgtaatcacatttatttcttttctgacagtgccgccatcatatcttccaggagcatttccgctccgcgcgggctgagtacgattttgccgcccgcatacgttttatttttcgtggtgatgtcgccggtcatcaaacatgccatgtttgcggcgtattttgtcaaaatgatattttcgccgtcggtatatatttcgagcgggtccttttcattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatgacgaactttggacaccatttacgacaattaagggaacggaaaaaactgaccgtcaatcaactggcgatgtattccggcgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgctaagttcccaagcccgcgacgatcagaaaattggcctgataactgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgccgacattaaaaagtggcggtaaaattcccccagcacatagccgccgttttgcgcgtaatacgtccaaagctgctgcagcacttccat

SEQ ID NO: 129 - DNA, 2391 bp, Artificial Sequence
  synthetic
ttatacaaagtaagcaagcgcaatcgttaagaaaaagaaaagcacggttaaaacgaccgtcatccggtgaagaatcaaatcaagtcctcttgctttctgctttccgaaaagctgctcggctccgccggaaatcgcgcctgataatccggcgcttttgccggattgcagtaaaacgacaacgattaatacgatacttacaatcactaataagaccgttaaaaatgcggccataacctacacctccagacaaactggctgacaatagttttattttacatgaaaagcaagcgcatgtcacgagcgtttcgaacagctttttttattttttcccagcgccggaataaggtatacaaaaaaagagcggctctgctccctttcctgcggaatatgtaatcacatttatttcttttctgacagtgccgccatcatatcttccaggagcatttccgctccgcgcgggctgagtacgattttgccgcccgcatacgttttatttttcgtggtgatgtcgccggtcatcaaacatgccatgtttgcggcgtattttgtcaaaatgatattttcgccgtcggtatatatttcgagcgggtccttttcattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgccgacattaaaaagtggcggtaaaattcccccagcacatagccgccgttttgcgcgtaatacgtccaaagctgctgcagcacttccat

SEQ ID NO: 130 - DNA, 2412 bp, Artificial Sequence
  synthetic
ttatacaaagtaagcaagcgcaatcgttaagaaaaagaaaagcacggttaaaacgaccgtcatccggtgaagaatcaaatcaagtcctcttgctttctgctttccgaaaagctgctcggctccgccggaaatcgcgcctgataatccggcgcttttgccggattgcagtaaaacgacaacgattaatacgatacttacaatcactaataagaccgttaaaaatgcggccataacctacacctccagacaaactggctgacaatagttttattttacatgaaaagcaagcgcatgtcacgagcgtttcgaacagctttttttattttttcccagcgccggaataaggtatacaaaaaaagagcggctctgctccctttcctgcggaatatgtaatcacatttatttcttttctgacagtgccgccatcatatcttccaggagcatttccgctccgcgcgggctgagtacgattttgccgcccgcatacgttttatttttcgtggtgatgtcgccggtcatcaaacatgccatgtttgcggcgtattttgtcaaaatgatattttcgccgtcggtatatatttcgagcgggtccttttcattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatgacgaactttggacaccatttacgacaattaagggaacggaaaaaactgaccgtcaatcaactggcgatgtattccggcgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggcaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgccgacattaaaaagtggcggtaaaattcccccagcacatagccgccgttttgcgcgtaatacgtccaaagctgctgcagcacttccat

SEQ ID NO: 131 - DNA, 1841 bp, Artificial Sequence
  synthetic
ttatacaaagtaagcaagcgcaatcgttaagaaaaagaaaagcacggttaaaacgaccgtcatccggtgaagaatcaaatcaagtcctcttgctttctgctttccgaaaagctgctcggctccgccggaaatcgcgcctgataatccggcgcttttgccggattgcagtaaaacgacaacgattaatacgatacttacaatcactaataagaccgttaaaaatgcggccataacctacacctccagacaaactggctgacaatagttttattttacatgaaaagcaagcgcatgtcacgagcgtttcgaacagctttttttattttttcccagcgccggaataaggtatacaaaaaaagagcggctctgctccctttcctgcggaatatgtaatcacatttatttcttttctgacagtgccgccatcatatcttccaggagcatttccgctccgcgcgggctgagtacgattttgccgcccgcatacgttttatttttcgtggtgatgtcgccggtcatcaaacatgccatgtttgcggcgtattttgtcaaaatgatattttcgccgtcggtatatatttcgagcgggtccttttcattgatattcagcaccctgcgcatttcgaccgggagaacgactctgccgagctcatcgattctccggacaatcccggtatttttcacgtttgaaaagcctccttttctcctttctttattgacttttgtcaacatctttataataaaagagatcttcaaattttttgttgaaatactgaatcatctttccgatcacaagttgtccgggcctcctttcgccatttaaaactctgctgagtgtcgccggggatacgccgatttcaatggcaagctgatttaaggagagattgtgttcaatcatgtactggagaacaaaatctcttttgatatgaatcttttttaccatgattactcccctttctaatctcttatgtttctttttatctacattgaacatatacgatttgttaacttttgtcaatacttttaccatccatatgtttcctataggcaatattcgtactaaaatattttataataagagattgcgaggttttggccatgacgaaccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgccgacattaaaaagtggcggtaaaattcccccagcacatagccgccgttttgcgcgtaatacgtccaaagctgctgcagcacttccat

SEQ ID NO: 132 - DNA, 1124 bp, Artificial Sequence
  synthetic
ttatacaaagtaagcaagcgcaatcgttaagaaaaagaaaagcacggttaaaacgaccgtcatccggtgaagaatcaaatcaagtcctcttgctttctgctttccgaaaagctgctcggctccgccggaaatcgcgcctgataatccggcgcttttgccggattgcagtaaaacgacaacgattaatacgatacttacaatcactaataagaccgttaaaaatgcggccataacctacacctccagacaaactggctgacaatagttttattttacatgaaaagcaagcgcatgtcacgagcgtttcgaacagctttttttattttttcccagcgccggaataaggtatacaaaaaaagagcggctctgctccctttcctgcggaatatgtaatcacataaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatgacgagcgcggttctgagccccgccataatgaccgacaaggcgaggggaagctccaccatccggagcacttgaaatttcgtcatgcccatcgccttccctgattcaagataggcatgctcgatgctggcgattcccgtatatgtgtttcgaatgatcggcaacagcgaatacaaaaacaatgaaagaatcaccgtgtttgcgccgagccccatgacaagcatcaagacggcgagcatcgccagcgccggaaccgtttgaatgacattagtgatggaaaagacccatttgctgattttacggtatctggcgatgaaaatgccggccgggatgccgacgacggcggcgaacaatacgccgtatgccgacattaaaaagtggcggtaaaattcccccagcacatagccgccgttttgcgcgtaatacgtccaaagctgctgcagcacttccat

(19) United States
(12) Patent Application Publication
(10) Pub. No.: US 2022/0282234 A1
(43) Pub. Date: Sep. 8, 2022

(54) COMPOSITIONS AND METHODS FOR INCREASED PROTEIN PRODUCTION IN BACILLUS LICHENFORMIS

(71) Applicant: DANISCO US INC., Palo Alto, CA (US)

(72) Inventors: Ryan L. FRISCH, Palo Alto, CA (US); Hongxian HE, Palo Alto, CA (US)

(21) Appl. No.: 17/635,349

(22) PCT Filed: Aug. 10, 2020
(86) PCT No.: PCT/US2020/045618

§ 371 Date: Feb. 14, 2022

Related U.S. Application Data
(60) Provisional application No. 62/886,571, filed on Aug. 14, 2019.

(51) Int. Cl. C12N 9/28; C12N 1/20; C12N 15/11; C12N 9/22; C12N 15/75; C07K 14/32

(52) U.S. Cl. CPC C12N 9/2417; C12N 1/205; C12N 15/11; C12N 9/22; C12N 15/75; C07K 14/32; C12R 2001/10

(57) ABSTRACT

The present disclosure is generally related to compositions and methods for constructing and obtaining Bacillus licheniformis cells having increased protein production phenotypes. Thus, certain embodiments are related to modified B. licheniformis cells derived from parental B. licheniformis cells. Certain embodiments are related to modified B. licheniformis cells comprising a modified rghR locus. Certain embodiments are related to modified B. licheniformis cells having a modified rghR locus and comprising an increased protein productivity phenotype. In certain other embodiments, modified B. licheniformis cells having a modified rghR locus produce a reduced amount of red pigment. In certain other embodiments, modified B. licheniformis cells comprise an increased protein productivity phenotype and produce a reduced amount of red pigment.

### CROSS-REFERENCE TO RELATED APPLICATIONS

The instant application claims priority to U.S. Provisional Patent Application No. 62/886,571, filed Aug. 14, 2019, which is hereby incorporated by reference in its entirety.

### FIELD

The present disclosure is generally related to the fields of bacteriology, microbiology, genetics, molecular biology, enzymology, industrial protein production the like. More particularly, the present disclosure is related to compositions and methods for obtaining Bacillus licheniformis strains having increased protein production capabilities.

### REFERENCE TO A SEQUENCE LISTING

The contents of the electronic submission of the text file Sequence Listing, named “NB41514-WO-PCT_SequenceListing.txt” was created on Jun. 23, 2020 and is 316 KB in size, which is hereby incorporated by reference in its entirety.

### BACKGROUND

Gram-positive bacteria such as Bacillus subtilis, Bacillus licheniformis and Bacillus amyloliquefaciens are frequently used as microbial factories for the production of industrial relevant proteins, due to their excellent fermentation properties and high yields (e.g., up to 25 grams per liter culture; Van Dijl and Hecker, 2013). For example, B. subtilis is well known for its production of α-amylases (Jensen et al., 2000; Raul et al., 2014) and proteases (Brode et al., 1996) necessary for food, textile, laundry, medical instrument cleaning, pharmaceutical industries and the like (Westers et al., 2004). Because these non-pathogenic Gram-positive bacteria produce proteins that completely lack toxic by-products (e.g., lipopolysaccharides; LPS, also known as endotoxins) they have obtained the “Qualified Presumption of Safety” (QPS) status of the European Food Safety Authority, and many of their products gained a “Generally Recognized As Safe” (GRAS) status from the US Food and Drug Administration (Olempska-Beer et al., 2006; Earl et al., 2008; Caspers et al., 2010).

Thus, the production of proteins (e.g., enzymes, antibodies, receptors, etc.) in microbial host cells is of particular interest in the biotechnological arts. Likewise, the optimization of Bacillus host cells for the production and secretion of one or more protein(s) of interest is of high relevance, particularly in the industrial biotechnology setting, wherein small improvements in protein yield are quite significant when the protein is produced in large industrial quantities. More particularly, B. licheniformis is a Bacillus species host cell of high industrial importance, and as such, the ability to modify and engineer B. licheniformis host cells for enhanced/increased protein expression/production is highly desirable for construction of new and improved B. licheniformis production strains. The present disclosure is therefore related to the highly desirable and unmet need for obtaining and constructing B. licheniformis cells (e.g., protein production host cells) having increased protein production capabilities.

### SUMMARY

The present disclosure is generally related to compositions and methods for constructing and obtaining Bacillus licheniformis cells (strains) having increased protein production phenotypes. More particularly, certain embodiments are related to modified Bacillus licheniformis cells derived from parental B. licheniformis cells comprising a native rghR (chromosomal) locus, wherein the modified cells comprise at least one modification of the rghR locus selected from the group consisting of (i) a modified rghR1 gene, (ii) a modified rghR2 gene, (iii) a modified rghR1 gene and modified rghR2 gene, and (iv) a modified rghR1 gene, a modified rghR2 gene, a modified yvzC gene and a modified Bli3644 gene, wherein the modified cell produces an increased amount of a protein of interest (relative to the parental cell when cultivated under the same conditions).

In certain embodiments, the modified rghR1 gene comprises a genetic modification which mutates, disrupts, partially deletes, or completely deletes the encoded RghR1 protein and/or the modified rghR1 gene comprises a genetic modification which mutates, disrupts, partially deletes, or completely deletes a 5′-UTR sequence and/or a 3′-UTR sequence of the rghR1 gene, wherein the modified rghR1 gene does not express or produce the encoded RghR1 protein.

In certain embodiments, the modified rghR2 gene comprises a genetic modification which mutates, disrupts, partially deletes, or completely deletes the encoded RghR2 protein and/or the modified rghR2 gene comprises a genetic modification which mutates, disrupts, partially deletes, or completely deletes a 5′-UTR sequence and/or a 3′-UTR of the rghR2 gene, wherein the modified rghR2 gene does not express or produce the encoded RghR2 protein.

In certain embodiments, the modified yvzC gene comprises a genetic modification which mutates, disrupts, partially deletes, or completely deletes the encoded YvzC protein and/or the modified yvzC gene comprises a genetic modification which mutates, disrupts, partially deletes, or completely deletes a 5′-UTR sequence and/or a 3′-UTR of the yvzC gene, wherein the modified yvzC gene does not express or produce the encoded YvzC protein.

In certain embodiments, the modified Bli3644 gene comprises a genetic modification which mutates, disrupts, partially deletes, or completely deletes the encoded Bli3644 protein and/or the modified Bli3644 gene comprises a genetic modification which mutates, disrupts, partially deletes, or completely deletes a 5′-UTR sequence and/or a 3′-UTR of the Bli3644 gene, wherein the modified Bli3644 gene does not express or produce the encoded Bli3644 protein.

Thus, in certain embodiments, a modified B. licheniformis cell comprising at least one genetic modification of the rghR locus comprises a modified rghR1 gene. In other embodiments, a modified B. licheniformis cell comprising at least one genetic modification of the rghR locus comprises a modified rghR2 gene. In other embodiments, a modified B. licheniformis cell comprising at least one genetic modification of the rghR locus comprises a modified rghR1 gene and a modified rghR2 gene. In other embodiments, a modified B. licheniformis cell comprising at least one genetic modification of the rghR locus comprises a modified rghR1 gene, a modified rghR2 gene, a modified yvzC gene and a modified bli3644 gene. In certain other embodiments, a modified B. licheniformis cell comprises a deleted rghR locus. In certain other embodiments, the modified cells produce a reduced amount of red pigment (relative to the parental cell when cultivated under the same conditions). In yet other embodiments, the B. licheniformis cells comprise one or more expression cassettes encoding a protein of interest. In certain embodiments, the one or more expressions cassettes encode an amylase protein.

In other embodiments, the disclosure is related to modified B. licheniformis cells derived from parental B. licheniformis cells comprising a native rghR2 gene, wherein the modified cells comprise at least one genetic modification which mutates, disrupts, partially deletes, or completely deletes the rghR2 gene, wherein the modified cells produce a reduced amount of red pigment (relative to the parental cell when cultivated under the same conditions). In certain embodiments, the cells comprise one or more expression cassettes encoding a protein of interest. In particular embodiments, the one or more expressions cassettes encode an amylase protein. In certain other embodiments, the modified cells produce an increased amount of a protein of interest (relative to the parental cell when cultivated under the same conditions).

Thus, certain other embodiments of the disclosure are related to methods for producing an increased amount of a protein of interest in modified B. licheniformis cells comprising (a) obtaining a B. licheniformis cell and genetically modifying at least one gene of the rghR locus selected from the group consisting of (i) a rghR1 gene, (ii) a rghR2 gene, (iii) ayvzC gene and (iv) a Bli3644 gene, or a combination thereof, and (b) fermenting the modified cell of step (a) under suitable conditions for the production of a protein of interest, wherein the modified cell produces an increased amount of the protein of interest (relative to the parental cell when cultivated under the same conditions).

In certain embodiments of the method, a modified rghR1 gene comprises a genetic modification which mutates, disrupts, partially deletes, or completely deletes the encoded RghR1 protein, and/or a modified rghR1 gene comprises a genetic modification which mutates, disrupts, partially deletes, or completely deletes a 5′-UTR sequence and/or a 3′-UTR sequence of the rghR1 gene, wherein the modified rghR1 gene does not express the encoded RghR1 protein.

In other embodiments, a modified rghR2 gene comprises a genetic modification which mutates, disrupts, partially deletes, or completely deletes the encoded RghR2 protein, and/or a modified rghR2 gene comprises a genetic modification which mutates, disrupts, partially deletes, or completely deletes a 5′-UTR sequence and/or a 3′-UTR of the rghR2 gene, wherein the modified rghR2 gene does not express the encoded RghR2 protein.

In another embodiment, a modified yvzC gene comprises a genetic modification which mutates, disrupts, partially deletes, or completely deletes the encoded YvzC protein, and/or a modified yvzC gene comprises a genetic modification which mutates, disrupts, partially deletes, or completely deletes a 5′-UTR sequence and/or a 3′-UTR of the yvzC gene, wherein the modified yvzC gene does not express the encoded YvzC protein.

In certain other embodiments of the method, a modified Bli3644 gene comprises a genetic modification which mutates, disrupts, partially deletes, or completely deletes the encoded Bli3644 protein, and/or a modified Bli3644 gene comprises a genetic modification which mutates, disrupts, partially deletes, or completely deletes a 5′-UTR sequence and/or a 3′-UTR of the Bli3644 gene, wherein the modified Bli3644 gene does not express the encoded Bli3644 protein.

In another embodiment of the method, the cells comprise one or more expression cassettes encoding a protein of interest. In certain embodiments, the one or more expressions cassettes encode an amylase protein. In another embodiment of the method, the modified B. licheniformis cells produce a reduced amount of red pigment.

In other embodiments, the disclosure is related to a method for producing a protein of interest in modified B. licheniformis cells, wherein the modified cells produce a reduced amount of red pigment during fermentation, the method comprising (a) obtaining a B. licheniformis cell and genetically modifying the rghR2 gene therein, and (b) fermenting the modified cell under suitable conditions for the production of a protein of interest, wherein the modified cell produces a reduced red pigment (relative to the parental cell when cultivated under the same conditions). In certain embodiments, a modified rghR2 gene comprises a genetic modification which mutates, disrupts, partially deletes, or completely deletes the encoded RghR2 protein. I other embodiments, the cells comprise one or more expression cassettes encoding a protein of interest. In certain embodiments, the one or more expressions cassettes encode an amylase protein. In other embodiments, the modified cells produce an increased amount of the protein of interest (relative to the parental cell when cultivated under the same conditions).

### BRIEF DESCRIPTION OF THE DRAWINGS

FIG. 1 is a schematic diagram of the B. licheniformis chromosomal “rghR locus”, wherein the wild-type rghR locus

FIG. 1A comprises the rghR1 gene (white arrow), rghR2 gene (black arrow), yvzC gene (grey arrow) and Bli3644 gene (stripe filled arrow). As further described in the Example section below,

FIG. 1B shows a modified rghR locus comprising a rghR2ₛₜₒₚ allele (white arrow, showing three (3) asterisks indicating stop codons), the native rghR1 gene (black arrow), native yvzC gene (grey arrow) and native Bli3644 gene (stripe filled arrow);

FIG. 1C shows a modified rghR locus comprising a deleted rghR1 (ΔrghR1) allele, the native rghR2 gene (white arrow), native yvzC gene (grey arrow) and native Bli3644 gene (stripe filled arrow);

FIG. 1D shows a rghR locus comprising a deleted rghR2 (ΔrghR2) allele, the native rghR1 gene (black arrow), native yvzC gene (grey arrow) and native Bli3644 gene (stripe filled arrow);

FIG. 1E shows a modified rghR locus comprising a deleted rghR2 (ΔrghR2) allele, a deleted rghR1 (ΔrghR1) allele, the native yvzC gene (grey arrow) and native Bli3644 gene (stripe filled arrow); and

FIG. 1F shows a modified (empty) rghR locus comprising a deletion of the rghR2, rghR1, yvzC and Bli3644 alleles (ΔrghR2/ΔrghR1/AyvzC/A3644).

### BRIEF DESCRIPTION OF THE BIOLOGICAL SEQUENCES

SEQ ID NO: 1 is the amino acid sequence of a S. pyogenes Cas9 protein.

SEQ ID NO: 2 is a nucleic acid encoding the Cas9 protein of SEQ ID NO: 1, wherein the nucleic acid sequence has been codon optimized for expression in a Bacillus host strain.

SEQ ID NO: 3 is an amino acid N-terminal nuclear localization sequence (NLS).

SEQ ID NO: 4 is an amino acid C-terminal nuclear localization sequence (NLS).

SEQ ID NO: 5 is a deca-histidine (His) tag amino acid sequence.

SEQ ID NO: 6 is a B. subtilis aprE promoter nucleic acid sequence.

SEQ ID NO: 7 is a synthetic terminator nucleic acid sequence.

SEQ ID NO: 8 is a forward primer nucleic acid sequence.

SEQ ID NO: 9 is a reverse primer nucleic acid sequence.

SEQ ID NO: 10 is the pKB320 backbone nucleic acid sequence.

SEQ ID NO: 11 is the nucleic acid sequence of plasmid pKB320.

SEQ ID NO: 12 is a forward primer nucleic acid sequence.

SEQ ID NO: 13 is a reverse primer nucleic acid sequence.

SEQ ID NO: 14 is a reverse sequencing primer.

SEQ ID NO: 15 is a reverse sequencing primer.

SEQ ID NO: 16 is a forward sequencing primer.

SEQ ID NO: 17 is a forward sequencing primer.

SEQ ID NO: 18 is a forward sequencing primer.

SEQ ID NO: 19 is a forward sequencing primer.

SEQ ID NO: 20 is a forward sequencing primer.

SEQ ID NO: 21 is a forward sequencing primer.

SEQ ID NO: 22 is a forward sequencing primer.

SEQ ID NO: 23 is a reverse sequencing primer.

SEQ ID NO: 24 is a forward sequencing primer.

SEQ ID NO: 25 is the nucleic acid sequence of plasmid pRF694.

SEQ ID NO: 26 is the nucleic acid sequence of plasmid pRF801.

SEQ ID NO: 27 is the nucleic acid sequence of plasmid pRF806.

SEQ ID NO: 28 is a B. licheniformis target site 1 (TS1) nucleic acid sequence.

SEQ ID NO: 29 is a B. licheniformis target site 2 (TS2) nucleic acid sequence.

SEQ ID NO: 30 is a B. licheniformis serA open reading frame nucleic acid sequence.

SEQ ID NO: 31 is a B. licheniformis target site 1 (TS1) PAM nucleic acid sequence.

SEQ ID NO: 32 is a nucleic acid sequence encoding a B. licheniformis variable targeting (VT) site 1.

SEQ ID NO: 33 is a nucleic acid sequence encoding a Cas9 endonuclease recognition (CER) domain.

SEQ ID NO: 34 is a guide RNA (gRNA) nucleic acid sequence targeting site 1.

SEQ ID NO: 35 is a spac promoter nucleic acid sequence.

SEQ ID NO: 36 is a t0 terminator nucleic acid sequence.

SEQ ID NO: 37 is B. licheniformis serA1 homology arm 1 nucleic acid sequence.

SEQ ID NO: 38 is a synthetic serA1 homology arm 1 forward primer sequence.

SEQ ID NO: 39 is a synthetic serA1 homology arm 1 reverse primer sequence.

SEQ ID NO: 40 is B. licheniformis serA1 homology arm 2 nucleic acid sequence.

SEQ ID NO: 41 is a synthetic serA1 homology arm 2 forward primer sequence.

SEQ ID NO: 42 is a synthetic serA1 homology arm 2 reverse primer sequence.

SEQ ID NO: 43 is an expression cassette encoding the target site 1 (TS1) gRNA.

SEQ ID NO: 44 is a synthetic serA1 deletion editing template.

SEQ ID NO: 45 is a B. licheniformis rghR1 open reading frame nucleic acid sequence.

SEQ ID NO: 46 is a targeting site 2 (TS2) PAM nucleic acid sequence.

SEQ ID NO: 47 is a nucleic acid sequence encoding variable targeting (VT) site 2.

SEQ ID NO: 48 is a gRNA nucleic acid sequence targeting site 2.

SEQ ID NO: 49 is a B. licheniformis rghR1 homology arm 1 nucleic acid sequence.

SEQ ID NO: 50 is a synthetic rghR1 homology arm 1 forward sequence.

SEQ ID NO: 51 is a synthetic rghR1 homology arm 1 reverse sequence.

SEQ ID NO: 52 is a B. licheniformis rghR1 homology arm 2 nucleic acid sequence.

SEQ ID NO: 53 is a synthetic rghR1 homology arm 2 forward sequence.

SEQ ID NO: 54 is a synthetic rghR1 homology arm 2 reverse sequence.

SEQ ID NO: 55 is a synthetic nucleic acid expression cassette encoding target site 2 (TS2) gRNA.

SEQ ID NO: 56 is a synthetic rghR1 deletion editing template sequence.

SEQ ID NO: 57 is the amino acid sequence of a Cas9 (Y155H) variant protein.

SEQ ID NO: 58 is a Cas9 (Y155H) forward primer sequence.

SEQ ID NO: 59 is a Cas9 (Y155H) reverse primer sequence.

SEQ ID NO: 60 is the nucleic acid sequence of plasmid pRF827.

SEQ ID NO: 61 is an expression cassette encoding the variant Cas9 (Y155H) protein.

SEQ ID NO: 62 is the nucleic acid sequence of plasmidpRF856.

SEQ ID NO: 63 is a synthetic Cas9 (Y155H) fragment nucleic acid sequence.

SEQ ID NO: 64 is Cas9 (Y155H) fragment forward primer sequence.

SEQ ID NO: 65 is Cas9 (Y155H) fragment reverse primer sequence.

SEQ ID NO: 66 is the nucleic acid sequence of plasmid pRF694.

SEQ ID NO: 67 is a pRF694 fragment nucleic acid sequence.

SEQ ID NO: 68 is a pRF694 fragment forward primer sequence.

SEQ ID NO: 69 is a pRF694 fragment reverse primer sequence.

SEQ ID NO: 70 is the nucleic acid sequence of plasmid pRF869.

SEQ ID NO: 71 is a B. licheniformis rghR2 open reading frame nucleic acid sequence.

SEQ ID NO: 72 is a synthetic rghR2ₛₜₒₚ fragment nucleic acid sequence.

SEQ ID NO: 73 is a synthetic rghR2ₛₜₒₚ editing template sequence.

SEQ ID NO: 74 is a rghR2 gRNA expression cassette.

SEQ ID NO: 75 is a synthetic fragment forward primer.

SEQ ID NO: 76 is a synthetic fragment reverse primer.

SEQ ID NO: 77 is the nucleic acid sequence of the pRF862 backbone.

SEQ ID NO: 78 is a pRF862 backbone forward primer.

SEQ ID NO: 79 is a pRF862 backbone reverse primer.

SEQ ID NO: 80 is the nucleic acid sequence of plasmid pRF874.

SEQ ID NO: 81 is a pRF874 target site and PAM nucleic acid sequence.

SEQ ID NO: 82 is a pRF874 editing template.

SEQ ID NO: 83 is the nucleic acid sequence of plasmid pRF879.

SEQ ID NO: 84 is a pRF879 target site and PAM nucleic acid sequence.

SEQ ID NO: 85 is a pRF879 editing template.

SEQ ID NO: 86 is the nucleic acid sequence of plasmid pRF899.

SEQ ID NO: 87 is a pRF899 and pRF901 target site and PAM nucleic acid sequence.

SEQ ID NO: 88 is a pRF899 editing template.

SEQ ID NO: 89 is the nucleic acid sequence of plasmid pRF901.

SEQ ID NO: 90 is a pRF901 editing template.

SEQ ID NO: 91 is a wild-type rghR2 locus nucleic acid sequence.

SEQ ID NO: 92 is a lysA open reading frame nucleic acid sequence.

SEQ ID NO: 93 is a serA_α-amylase expression cassette.

SEQ ID NO: 94 is synthetic p3 promoter nucleic acid sequence.

SEQ ID NO: 95 is aprE 5-untranslated region (UTR) nucleic acid sequence.

SEQ ID NO: 96 is a nucleic acid sequence encoding an amyL signal sequence.

SEQ ID NO: 97 is a nucleic acid sequence encoding an α-amylase protein.

SEQ ID NO: 98 is a nucleic acid sequence encoding an amyL terminator sequence.

SEQ ID NO: 99 is a synthetic amyL α-amylase expression cassette.

SEQ ID NO: 100 is a B. licheniformis amyL promoter sequence.

SEQ ID NO: 101 is apBl.comKnucleic acid sequence.

SEQ ID NO: 102 is a nucleic acid sequence encoding a spectinomycin marker.

SEQ ID NO: 103 is a B. licheniformis xy1R open reading frame.

SEQ ID NO: 104 is B. licheniformis xy1A promoter sequence.

SEQ ID NO: 105 is a nucleic acid sequence encoding a ComK protein.

SEQ ID NO: 106 is a forward primer sequence.

SEQ ID NO: 107 is a reverse primer sequence.

SEQ ID NO: 108 is a B. licheniformis rghR2 targeted region nucleic acid sequence.

SEQ ID NO: 109 is a synthetic rghR2_{Stop} nucleic acid sequence.

SEQ ID NO: 110 is a forward primer sequence.

SEQ ID NO: 111 is a forward primer sequence.

SEQ ID NO: 112 is a reverse primer sequence.

SEQ ID NO: 113 is a B. licheniformis native rghR1 sequence.

SEQ ID NO: 114 is a rghR1 deletion PCR product.

SEQ ID NO: 115 is a forward primer sequence.

SEQ ID NO: 116 is a reverse primer sequence.

SEQ ID NO: 117 is a B. licheniformis native rghR2 PCR product.

SEQ ID NO: 118 is a rghR2 deletion PCR product.

SEQ ID NO: 119 is a forward primer sequence.

SEQ ID NO: 120 is a reverse primer sequence.

SEQ ID NO: 121 is a B. licheniformis native rghR1 rghR2 PCR product.

SEQ ID NO: 122 is rghR1 rghR2 deletion PCR product.

SEQ ID NO: 123 is a forward primer sequence.

SEQ ID NO: 124 is a reverse primer sequence.

SEQ ID NO: 125 is B. licheniformis native locus PCR product

SEQ ID NO: 126 is a synthetic locus deletion PCR product.

SEQ ID NO: 127 is a B. licheniformis LDN143 strain rghR2 locus nucleic acid sequence.

SEQ ID NO: 128 is a B. licheniformis BF314 strain rghR2 locus nucleic acid sequence.

SEQ ID NO: 129 is a B. licheniformis BF324 strain rghR2 locus nucleic acid sequence.

SEQ ID NO: 130 is a B. licheniformis BF377 strain rghR2 locus nucleic acid sequence.

SEQ ID NO: 131 is a B. licheniformis BF389 strain rghR2 locus nucleic acid sequence.

SEQ ID NO: 132 is a B. licheniformis BF391 strain rghR2 locus nucleic acid sequence.

### DETAILED DESCRIPTION

The present disclosure is generally related to compositions and methods for constructing and obtaining Bacillus licheniformis cells (strains) having increased protein production phenotypes. Thus, certain embodiments are related to modified B. licheniformis cells derived from parental B. licheniformis cells. In certain embodiments, a modified B. licheniformis cell comprises a modified rghR locus, wherein the parental cell from which it was derived comprises a wild-type rghR locus. In certain embodiments, a modified B. licheniformis cell having a modified rghR locus comprises an increased protein productivity phenotype. In certain other embodiments, a modified B. licheniformis cell having a modified rghR locus produces a reduced amount of red pigment. In certain other embodiments, a modified B. licheniformis cell comprises an increased protein productivity phenotype and produces a reduced amount of red pigment.

I. DEFINITIONS

In view of the modified Bacillus sp. cells of the disclosure and methods thereof described herein, the following terms and phrases are defined. Terms not defined herein should be accorded their ordinary meaning as used in the art.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described. All publications and patents cited herein are incorporated by reference in their entirety.

It is further noted that the claims may be drafted to exclude any optional element. As such, this statement is intended to serve as antecedent basis for use of such exclusive terminology as “solely,” “only”, “excluding”, “not including” and the like, in connection with the recitation of claim elements, or use of a “negative” limitation or proviso thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, “host cell” refers to a cell that has the capacity to act as a host or expression vehicle for a newly introduced DNA sequence. Thus, in certain embodiments of the disclosure, the host cells are for example Bacillus sp. cells or E. coli cells.

As used herein, “modified cells” refers to recombinant (host) cells that comprise at least one genetic modification which is not present in the “parental” host cell from which the modified cells are derived.

For example, in certain embodiments, a “parental” cell is altered (e.g., via one or more genetic modifications introduced into the parental cell) to generate a “modified” (daughter) cell thereof.

In certain embodiments, a parental cell may be referred to as a “control cell”, particularly when being compared with, or relative to, a “modified” Bacillus sp. (daughter) cell. As used herein, when the expression and/or production of a protein of interest (POI) in an “unmodified” (parental) cell (e.g., a control cell) is being compared to the expression and/or production of the same POI in a “modified” (daughter) cell, it will be understood that the “modified” and “unmodified” cells are grown/cultivated/fermented under the same conditions (e.g., the same conditions such as media, temperature, pH and the like).

As used herein, the “genus Bacillus” or “Bacillus sp.” cells include all species within the genus “Bacillus”” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

As used herein, the terms “wild-type” and “native” are used interchangeably and refer to genes, promoters, proteins, protein mixes, cells or strains, as found in nature.

As used herein, a “native B. licheniformis rghR2 gene” comprises a nucleotide sequence encoding a “native RghR2 protein” and a “variant-18-BP B. licheniformis rghR2 gene” comprises a nucleotide sequence encoding a “variant RghR2 protein” (RghR2aᵤp), described in PCT Publication No. WO2018/156705 (incorporated herein by reference in its entirety). For example, the variant-18-BP rghR2 gene (hereinafter, “rghR2_{dup}”), comprises a nucleotide sequence encoding a variant RghR2 protein (hereinafter, “RghR2aᵤp”), which variant RghR2aᵤp comprises a six (6) amino acid residue duplication/repeat (i.e., residues “AAASIR” are duplicated).

As used herein, a “native rghR1 gene” encodes a native RghR1 protein, a “native rghR2 gene” encodes a native RghR2 protein, a “native yvzC gene” encodes a native YvzC protein and a “native Bli3644 gene” a native Bli3644 protein.

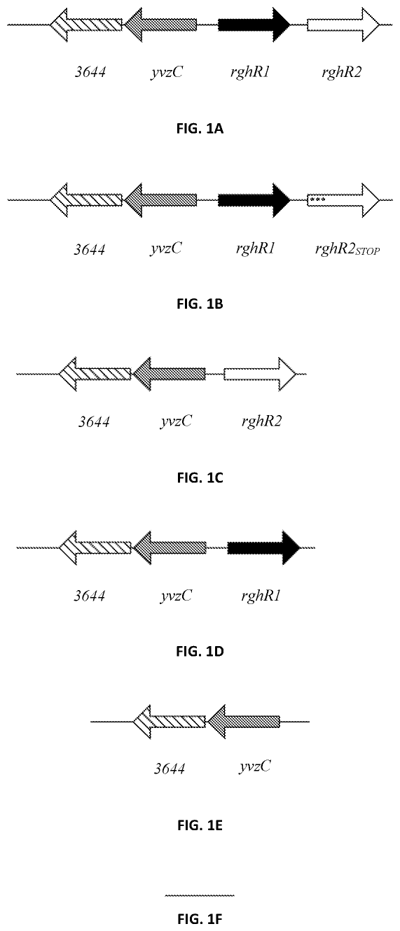

As used herein, a “native B. licheniformis (chromosomal) rghR locus” (hereinafter, “native rghR locus”) comprises a “native rghR1 gene”, a “native rghR2 gene”, a “native yvzC gene” and a “native Bli3644 gene”, as presented schematically in FIG. 1A.

As used herein, a parental B. licheniformis cell named “LDN143” comprises a native rghR locus.

As used herein, a “modified B. licheniformis (chromosomal) rghR locus” (hereinafter, “modified rghR locus”) comprises at least one genetic modification of a gene (or an open reading frame thereof) selected from rghR1, rghR2, yvzC and/or Bli3644, relative to the native rghR locus. In certain embodiments, a modified B. licheniformis cell comprising a modified rghR locus is derived from a parental B. licheniformis cell comprising a native rghR locus.

As used herein, a modified B. licheniformis (daughter) cell named “BF314” comprises a modified rghR locus comprising a native rghR1 gene, a modified rghR2 gene (named “rghRₛₜₒₚ”; comprising three (3) pre-mature stop codons), a native yvzC gene and a native Bli3644 gene, as presented schematically in FIG. 1B.

As used herein, a modified B. licheniformis (daughter) cell named “BF324” comprises a modified rghR locus comprising a deleted rghR1 gene (ΔrghR1), a native rghR2 gene, a native yvzC gene and a native Bli3644 gene, as presented schematically in FIG. 1C.

As used herein, a modified B. licheniformis (daughter) cell named “BF377” comprises a modified rghR locus comprising a native rghR1 gene, a deleted rghR2 gene (ΔrghR2), a native yvzC gene and a native Bli3644 gene, as presented schematically in FIG. 1D.

As used herein, a modified B. licheniformis (daughter) cell named “BF389” comprises a modified rghR locus comprising a deleted rghR1 gene (ΔrghR1), a deleted rghR2 gene (ΔrghR2), a native yvzC gene and a native Bli3644 gene, as presented schematically in FIG. 1E.

As used herein, a modified B. licheniformis (daughter) cell named “BF391” comprises a modified (empty) rghR locus comprising a deleted rghR1 gene (ΔrghR1), a deleted rghR2 gene (ΔrghR2), a deleted yvzC gene (AyvzC) and a deleted Bli3644 gene (ΔBli3644), as presented schematically in FIG. 1F.

As used herein, the term “equivalent positions” mean the amino acid residue positions after alignment with a specified polypeptide sequence.

The terms “modification” and “genetic modification” are used interchangeably and include: (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein. For example, as used herein a genetic modification includes, but is not limited to, a modification of one or more genes selected from the group consisting of rghR1, rghR2, yvzC, BLi3644, and the like.

As used herein, “disruption of a gene”, “gene disruption”, “inactivation of a gene” and “gene inactivation” are used interchangeably and refer broadly to any genetic modification that substantially prevents a host cell from producing a functional gene product (e.g., a protein). Exemplary methods of gene disruptions include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and any combinations and variations thereof which disrupt/inactivate the target gene(s) and substantially reduce or prevent the production of the functional gene product (i.e., a protein).

As defined herein, the combined term “expresses/produces”, as used in phrases such as “a modified (host) cell expresses/produces an increased amount of a protein of interest relative to the parental (host) cell”, the term (“expresses/produces”) is meant to include any steps involved in the expression and production of a protein of interest in host cell of the disclosure.

Thus, as used herein, “increasing” protein production or “increased” protein production is meant an increased amount of protein produced (e.g., an endogenous and/or heterologous POI). The protein may be produced inside the host cell, or secreted (or transported) into the culture medium. In certain embodiments, the protein of interest is produced (secreted) into the culture medium. Increased protein production may be detected for example, as higher maximal level of protein or enzymatic activity (e.g., such as protease activity, amylase activity, cellulase activity, hemicellulase activity and the like), or total extracellular protein produced as compared to the parental host cell.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein.

It is understood that the polynucleotides (or nucleic acid molecules) described herein include “genes”, “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter). Coding sequences can be operably linked to regulatory sequences in sense or antisense orientation.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in Bacillus. For example, in certain embodiments, the present disclosure is directed to a polynucleotide comprising a 5′ promoter (or 5′ promoter region, or tandem 5′ promoters and the like), wherein the promoter region is operably linked to a nucleic acid sequence encoding a protein of the disclosure. Thus, in certain embodiments, a functional promoter sequence controls the expression of a gene encoding a protein disclosed herein. In other embodiments, a functional promoter sequence controls the expression of a heterologous gene (or endogenous gene) encoding a protein of interest in a Bacillus cell, more particularly in a B. licheniformis host cell.

As defined herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure.

As defined herein, the term “introducing”, as used in phrases such as “introducing into a bacterial cell” or “introducing into a B. licheniformis cell at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection, conjugation and the like (e.g., see Ferrari et al., 1989).

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). For example, in certain embodiments of the disclosure, a parental B. licheniformis cell is modified (e.g., transformed) by introducing into the parental cell a polynucleotide construct comprising a promoter operably linked to a nucleic acid sequence encoding a protein of interest, thereby resulting in a modified B. licheniformis (daughter) host cell derived from the parental cell.

As used herein, “transformation” refers to introducing an exogenous DNA into a host cell so that the DNA is maintained as a chromosomal integrant or a self-replicating extra-chromosomal vector. As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, conjugation, and transduction (See e.g., Ferrari et al., 1989).

As used herein “an incoming sequence” refers to a DNA sequence that is introduced into the Bacillus chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein, “homology box” refers to a nucleic acid sequence, which is homologous to a sequence in the Bacillus chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the Bacillus chromosome a DNA construct is integrated and directs what part of the Bacillus chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, the marker can be an antimicrobial resistance marker (e.g., amp^{R}, phleo^{R}, spec^{R}, kan R, ery^{R}, tet^{R}, cmp^{R} andneo^{R} (see e.g., Guerot-Fleury, 1995; Palmeros et al., 2000; and Trieu-Cuot et al., 1983).

In some embodiments, the present invention provides a chloramphenicol resistance gene (e.g., the gene present on pC194, as well as the resistance gene present in the Bacillus licheniformis genome). This resistance gene is particularly useful in the present invention, as well as in embodiments involving chromosomal amplification of chromosomally integrated cassettes and integrative plasmids (see e.g., Albertini and Galizzi, 1985; Stahl and Ferrari, 1984). Other markers useful in accordance with the invention include, but are not limited to auxotrophic markers, such as serine, lysine, tryptophan; and detection markers, such as β-galactosidase or fluorescent proteins.

As defined herein, a host cell “genome”, a bacterial (host) cell “genome”, or a B. licheniformis (host) cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously or can integrate into a chromosome of a host organism).

An “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). In some embodiments the targeting vectors include elements to increase homologous recombination with the chromosome including but not limited to RNA-guided endonucleases, DNA-guided endonucleases, and recombinases. The ends can be closed such that the targeting vector forms a closed circle, such as, for example, insertion into a vector.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

As used herein, the term “protein of interest” or “POI” refers to a polypeptide of interest that is desired to be expressed in a Bacillus sp. host cell, wherein the POI is preferably expressed at increased levels. Thus, as used herein, a POI may be an enzyme, a substrate-binding protein, a surface-active protein, a structural protein, a receptor protein, and the like. In certain embodiments, a modified cell of the disclosure produces an increased amount of a heterologous POI or an increased amount of an endogenous POI, relative to the parental cell. In particular embodiments, an increased amount of a POI produced by a modified cell of the disclosure is at least a 0.5% increase, at least a 1.0% increase, at least a 5.0% increase, or a greater than 5.0% increase, relative to the parental cell.

Similarly, as defined herein, a “gene of interest” or “GOI” refers a nucleic acid sequence (e.g., a polynucleotide, a gene or an ORF) which encodes a POI. A “gene of interest” encoding a “protein of interest” may be a naturally occurring gene, a mutated gene or a synthetic gene.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof).

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide.

Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence. As used herein, a “variant” polynucleotide refers to a polynucleotide encoding a variant polypeptide, wherein the “variant polynucleotide” has a specified degree of sequence homology/identity with a parent polynucleotide, or hybridizes with a parent polynucleotide (or a complement thereof) under stringent hybridization conditions. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As defined herein, an “endogenous gene” refers to a gene in its natural location in the genome of an organism.

As defined herein, a “heterologous” gene, a “non-endogenous” gene, or a “foreign” gene refer to a gene (or ORF) not normally found in the host organism, but that is introduced into the host organism by gene transfer. As used herein, the term “foreign” gene(s) comprise native genes (or ORFs) inserted into a non-native organism and/or chimeric genes inserted into a native or non-native organism.

As defined herein, a “heterologous” nucleic acid construct or a “heterologous” nucleic acid sequence has a portion of the sequence which is not native to the cell in which it is expressed.

As defined herein, a “heterologous control sequence”, refers to a gene expression control sequence (e.g., a promoter or enhancer) which does not function in nature to regulate (control) the expression of the gene of interest. Generally, heterologous nucleic acid sequences are not endogenous (native) to the cell, or a part of the genome in which they are present, and have been added to the cell, by infection, transfection, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence.

The signal sequence may be endogenous or exogenous. A signal sequence is normally absent from the mature protein. A signal sequence is typically cleaved from the protein by a signal peptidase after the protein is transported.

The term “derived” encompasses the terms “originated” “obtained,” “obtainable,” and “created,” and generally indicates that one specified material or composition finds its origin in another specified material or composition, or has features that can be described with reference to the another specified material or composition.

As used herein, the term “homology” relates to homologous polynucleotides or polypeptides. If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. Whether two polynucleotide or polypeptide sequences have a sufficiently high degree of identity to be homologous as defined herein, can suitably be investigated by aligning the two sequences using a computer program known in the art, such as “GAP” provided in the GCG program package (Program Manual for the Wisconsin Package, Version 8, August 1994, Genetics Computer Group, 575 Science Drive, Madison, Wis., USA 53711) (Needleman and Wunsch, (1970). Using GAP with the following settings for DNA sequence comparison: GAP creation penalty of 5.0 and GAP extension penalty of 0.3.

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “specific productivity” is total amount of protein produced per cell per time over a given time period.

As defined herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, the term “ComK polypeptide” is defined as the product of a comK gene; a transcription factor that acts as the final auto-regulatory control switch prior to competence development; involved with activation of the expression of late competence genes involved in DNA-binding and uptake and in recombination (Liu and Zuber, 1998, Hamoen et al., 1998).

As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes).

As used herein, “orthologue” and “orthologous genes” refer to genes in different species that have evolved from a common ancestral gene (i.e., a homologous gene) by speciation. Typically, orthologues retain the same function during the course of evolution. Identification of orthologues finds use in the reliable prediction of gene function in newly sequenced genomes.

As used herein, “paralog” and “paralogous genes” refer to genes that are related by duplication within a genome. While orthologues retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

As used herein, “homology” refers to sequence similarity or identity, with identity being preferred.

This homology is determined using standard techniques known in the art (see e.g., Smith and Waterman, 1981; Needleman and Wunsch, 1970; Pearson and Lipman, 1988; programs such as GAP, BESTFIT, FASTA, and TFASTA in the Wisconsin Genetics Software Package (Genetics Computer Group, Madison, Wis.) and Devereux et. al., 1984).

As used herein, the term “hybridization” refers to the process by which a strand of nucleic acid joins with a complementary strand through base pairing, as known in the art. A nucleic acid sequence is considered to be “selectively hybridizable” to a reference nucleic acid sequence if the two sequences specifically hybridize to one another under moderate to high stringency hybridization and wash conditions.

Hybridization conditions are based on the melting temperature (Tₘ) of the nucleic acid binding complex or probe. For example, “maximum stringency” typically occurs at about Tₘ 5° C. (5° below the Tₘ of the probe); “high stringency” at about 5-10° C. below the Tₘ; “intermediate stringency” at about 10-20° C. below the Tₘ of the probe; and “low stringency” at about 20-25° C. below the Tₘ.

Functionally, maximum stringency conditions may be used to identify sequences having strict identity or near-strict identity with the hybridization probe; while an intermediate or low stringency hybridization can be used to identify or detect polynucleotide sequence homologs. Moderate and high stringency hybridization conditions are well known in the art. An example of high stringency conditions includes hybridization at about 42° C. in 50% formamide, 5×SSC, 5×Denhardt's solution, 0.5% SDS and 100 pg/ml denatured carrier DNA, followed by washing two times in 2×SSC and 0.5% SDS at room temperature (RT) and two additional times in 0. 1×SSC and 0.5% SDS at 42° C. An example of moderate stringent conditions including overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/ml denatured sheared salmon sperm DNA, followed by washing the filters in 1×SSC at about 37-50° C. Those of skill in the art know how to adjust the temperature, ionic strength, etc. as necessary to accommodate factors such as probe length and the like.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. “Recombination”, “recombining” or generating a “recombined” nucleic acid is the assembly of two or more nucleic acid fragments wherein the assembly gives rise to a chimeric gene.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the Bacillus chromosome. These sequences direct where in the Bacillus chromosome the new construct gets integrated and what part of the Bacillus chromosome will be replaced by the incoming sequence. In other embodiments, the 5′ and 3′ ends of a selective marker are flanked by a polynucleotide sequence comprising a section of the inactivating chromosomal segment. In some embodiments, a flanking sequence is present on only a single side (either 3′ or 5′), while in other embodiments, it is present on each side of the sequence being flanked. In some embodiments, the homology boxes are directly flanking each other and lacking an intervene sequence (e.g. for genes D-E-F the construct D-F) such that if the construct recombines within the genome gene E will be removed from the genome.

II. BACILLUS LICHENIFORMIS RGHR LOCUS

The Bacillus subtilis yvaN gene has been identified as a repressor of rapG, rapH and rapD genes, and renamed “rghR”, (i.e., rapG and rapH Repressor; Hayashi et al., 2006; Ogura & Fujita, 2007). For example, the B. licheniformis rghR locus encodes two (2) homologs of the B. subtilis RghR/YvaO (transcriptional regulator), which are named “RghR1” and “RghR2”. Upstream (5′) of the B. licheniformis rghR1 gene (e.g., see, FIG. 1A) are two (2) additional genes, yvzC (Bli3645) and Bli3644, encoding transcriptional regulatory proteins YvzC and Bli3644, respectively. More particularly, as generally defined above, the native B. licheniformis rghR (chromosomal) locus comprises a native rghR1 gene, a native rghR2 gene, a native yvzC gene and a native Bli3644 gene, as shown in FIG. 1A. For example, PCT Publication No. WO2018/156705 discloses a mutant B. licheniformis strain comprising a mutated rghR2 gene having a nucleotide sequence encoding a variant RghR2 protein named “RghR2aᵤp” (i.e., comprising a six amino acid repeat of “AAASIR”). As generally described in PCT Publication No. WO2018/156705, deletion of this eighteen (18) bp duplication from the rghR2aᵤp sequence (i.e., yielding allele rghR2resₜ) resulted in a decrease in biomass with a concomitant increase in heterologous protein production.

As described herein and the Examples section below, Applicant further designed, constructed and tested modified B. licheniformis cells to evaluate the rghR locus, and identify B. licheniformis cells having enhanced protein production (or other beneficial) phenotypes. More particularly, in the instant Examples, a parental B. licheniformis cell named LDN143, comprising a native rghR locus (FIG. 1A) with deletions of the serA and lysA genes and comprising two (2) heterologous α-amylase expression cassettes, was evaluated against modified B. licheniformis (daughter) cells (i.e., derived from LDN143 parent) comprising a modified rghR locus. Thus, the modified B. licheniformis (daughter) cells described herein were constructed with a series of modified rghR locus alleles, which were introduced into the parental B. licheniformis cell (LDN143).

More specifically, the following B. licheniformis (daughter) cells derived from the LDN143 parent were constructed, comprising one of the following modified rghR loci: B. licheniformis cell BF314, comprising a native rghR1 gene, a modified rghR2 gene (rghR2ₛₜₒₚ), a native yvzC gene and a native Bli3644 gene (FIG. 1B), B. licheniformis cell BF324, comprising a deleted rghR1 gene (ΔrghR1), a native rghR2 gene (rghR2ₛₜₒₚ), a native yvzC gene and a native Bli3644 gene (FIG. 1C), B. licheniformis cell BF377, comprising a native rghR1 gene, a deleted rghR2 gene (ΔrghR2), a native yvzC gene and a native Bli3644 gene (FIG. 1D), B. licheniformis cell BF389, comprising a deleted rghR1 gene (ΔrghR1), a deleted rghR2 gene (ΔrghR2), a native yvzC gene and a native Bli3644 gene (FIG. 1E), and B. licheniformis cell BF391, comprising a deleted rghR1 gene (ΔrghR1), a deleted rghR2 gene (ΔrghR2), a deleted yvzC gene (ΔyvzC) and a deleted Bli3644 gene (ΔB1i3644) (FIG. 1F, empty rghR locus).

Thus, as described below in Example 4 (e.g., see TABLE 20), the modified B licheniformis cells with mutations in the rghR locus demonstrate increased production phenotypes, with about 23-62% more amylase protein produced than the comparable parental cell (LDN143), which is wild-type for the rghR locus. Certain embodiments of the disclosure are therefore related to such modified Bacillus cells having a modified rghR locus and comprising an increased protein productivity phenotype. Certain other embodiments are related to such compositions and methods for constructing and obtaining a modified Bacillus cell. Thus, certain other embodiments are related to the expression/production of endogenous and/or heterologous proteins of interest a modified Bacillus cell of the disclosure.

III. BACILLUS LICHENIFORMIS CELLS PRODUCING REDUCED AMOUNTS OF RED PIGMENT

As generally understood by one of skill in the art, Bacilli are well established as host systems for the production of native and recombinant proteins. However, certain Bacillus species (e.g., B. subtilis, B. cereus, B. licheniformis, etc.) are known to synthesize pulcherriminic acid that is derived from cyclo-L-leucyl-L-leucyl, wherein the pulcherriminic acid is secreted into the growth medium and chelates ferric iron (by a non-enzymatic reaction) to form an extracellular red pigment known as pulcherrimin (MacDonald, 1965; Uffen and Canale-Parola, 1972). Therefore, Bacillus sp. (host) cells producing pulcherrimin in an amount sufficient to form a visible red pigment (i.e., during fermentation/cultivation) generally require one or more pulcherrimin removal steps during the recovery and/or purification of the protein of interest, or the pulcherrimin (red pigment) may co-purify with the protein of interest.

For example, a Bacillus sp. host cell with a desirable phenotype (e.g., such as increased protein production) may not necessarily have the most desirable characteristics for successful fermentation, recovery and/or purification of the protein of interest produced by the host cell (e.g., such as a red pigment phenotype). Thus, certain genetic approaches to mitigate the production pulcherrimin in Bacillus cells have been described in the art, such as International PCT Publication No. WO2004/011609, describing deletions of a cypX gene and/or a yvmC gene in Bacillus as a means to reduce pulcherrimin production.

As described herein and the Examples section below, Applicant has identified a novel means to mitigate the production of red pigment (pulcherrimin) in Bacillus licheniformis cells. More specifically, as presented and described below in Example 5, an identified feature of the rghR locus is the transcriptional control of the operon responsible for producing the iron scavenging pigment pulcherriminic acid. As set forth in this example, the B. licheniformis cells BF314 (i.e., comprising a modified (rghR2ₛₜₒₚ) gene) and BF377 (i.e., comprising a deleted (ΔrghR2) gene) both demonstrate a decrease in the production of red pigment to about 30-50%, while several other mutations increased the production of pulcherrimin to about 10-20% (e.g., see TABLE 21), indicating that mutations in the rghR locus control the biosynthesis of pulcherriminic acid.

Certain embodiments of the disclosure are therefore related to such modified Bacillus cells having a modified rghR locus which produce a reduced amount of red pigment. Certain other embodiments are related to such compositions and methods for constructing and obtaining a modified Bacillus cell producing a reduced amount of red pigment. Thus, certain other embodiments are related to the expression/production of endogenous and/or heterologous proteins of interest a modified Bacillus cell of the disclosure.

IV. MOLECULAR BIOLOGY

As set forth above, certain embodiments of the disclosure are related to modified B. licheniformis cells derived from parental B. licheniformis cells comprising a native rghR locus. In particular embodiments, a modified B. licheniformis cell comprises a modified rghR locus. Thus, certain other embodiments are related to compositions and methods for genetically modifying a parental B. licheniformis cell to generate modified B. licheniformis (daughter) cell.

Certain embodiments are therefore related to methods for genetically modifying Bacillus cells, including, but not limited to, (a) the introduction, substitution, or removal of one or more nucleotides in a gene (or an ORF thereof), or the introduction, substitution, or removal of one or more nucleotides in a regulatory element required for the transcription or translation of the gene (or ORF thereof), (b) a gene disruption, (c) a gene conversion, (d) a gene deletion, (e) a gene down-regulation, (f) site specific mutagenesis and/or (g) random mutagenesis. For example, as used herein a genetic modification includes, but is not limited to, a modification of one or more genes selected from the group consisting of a B. licheniformis rghR1 gene, rghR2 gene, yvzC gene and Bli3644 gene.

Thus, in certain embodiments, a modified Bacillus cell of the disclosure is constructed by reducing or eliminating the expression of a gene set forth above, using methods well known in the art, for example, insertions, disruptions, replacements, or deletions. The portion of the gene to be modified or inactivated may be, for example, the coding region or a regulatory element required for expression of the coding region.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

In certain other embodiments a modified Bacillus cell is constructed by gene deletion to eliminate or reduce the expression of at least one of the aforementioned genes of the disclosure. Gene deletion techniques enable the partial or complete removal of the gene(s), thereby eliminating their expression, or expressing a non-functional (or reduced activity) protein product. In such methods, the deletion of the gene(s) may be accomplished by homologous recombination using a plasmid that has been constructed to contiguously contain the 5′ and 3′ regions flanking the gene. The contiguous 5′ and 3′ regions may be introduced into a Bacillus cell, for example, on a temperature-sensitive plasmid, such as pE194, in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is effected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers (see, e.g., Perego, 1993). Thus, a person of skill in the art (e.g., by reference to the rghR1, rghR2, yvzC, bli3644 (nucleic acid) sequences and the encoded protein sequences thereof), may readily identify nucleotide regions in the gene's coding sequence and/or the gene's non-coding sequence suitable for complete or partial deletion.

In other embodiments, a modified Bacillus cell of the disclosure is constructed by introducing, substituting, or removing one or more nucleotides in the gene or a regulatory element required for the transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art (e.g., see, Botstein and Shortle, 1985; Lo et al., 1985; Higuchi et al., 1988; Shimada, 1996; Ho et al., 1989; Horton et al., 1989 and Sarkar and Sommer, 1990). Thus, in certain embodiments, a gene of the disclosure is inactivated by complete or partial deletion.

In another embodiment, a modified Bacillus cell is constructed by the process of gene conversion (e.g., see Iglesias and Trautner, 1983). For example, in the gene conversion method, a nucleic acid sequence corresponding to the gene(s) is mutagenized in vitro to produce a defective nucleic acid sequence, which is then transformed into the parental Bacillus cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is effected by selection for the marker under conditions not permitting plasmid replication. Selection for a second recombination event leading to gene replacement is effected by examination of colonies for loss of the selectable marker and acquisition of the mutated gene (Perego, 1993). Alternatively, the defective nucleic acid sequence may contain an insertion, substitution, or deletion of one or more nucleotides of the gene, as described below.

In other embodiments, a modified Bacillus cell is constructed by established anti-sense techniques using a nucleotide sequence complementary to the nucleic acid sequence of the gene (Parish and Stoker, 1997). More specifically, expression of the gene by a Bacillus cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to RNA interference (RNAi), small interfering RNA (siRNA), microRNA (miRNA), antisense oligonucleotides, and the like, all of which are well known to the skilled artisan.

In other embodiments, a modified Bacillus cell is produced/constructed via CRISPR-Cas9 editing. For example, a gene encoding rghR1, rghR2, yvzC and/or Bli3644 can be disrupted (or deleted or down-regulated) by means of nucleic acid guided endonucleases, that find their target DNA by binding either a guide RNA (e.g., Cas9) and Cpfl or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete the gene. For example, the gene encoding the nucleic acid guided endonuclease (for this purpose Cas9 from S. pyogenes) or a codon optimized gene encoding the Cas9 nuclease is operably linked to a promoter active in the Bacillus cell and a terminator active in Bacillus cell, thereby creating a Bacillus Cas9 expression cassette. Likewise, one or more target sites unique to the gene of interest are readily identified by a person skilled in the art. For example, to build a DNA construct encoding a gRNA-directed to a target site within the gene of interest using Streptococcus pyogenes Cas9, the variable targeting domain (VT) will comprise nucleotides of the target site which are 5′ of the (PAM) proto-spacer adjacent motif (NGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a Bacillus expression cassette for the gRNA is created by operably linking the DNA encoding the gRNA to a promoter active in Bacillus cells and a terminator active in Bacillus cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500-bp 5′ of targeted gene can be fused to about 500-bp 3′ of the targeted gene to generate an editing template, which template is used by the Bacillus host's machinery to repair the DNA break generated by the RGEN.

The Cas9 expression cassette, the gRNA expression cassette and the editing template can be co-delivered to the cells using many different methods. The transformed cells are screened by PCR amplifying the target gene locus, by amplifying the locus with a forward and reverse primer. These primers can amplify the wild-type locus or the modified locus that has been edited by the RGEN. These fragments are then sequenced using a sequencing primer to identify edited colonies (e.g., see Examples section below).

In yet other embodiments, a modified Bacillus cell is constructed by random or specific mutagenesis using methods well known in the art, including, but not limited to, chemical mutagenesis (see, e.g., Hopwood, 1970) and transposition (see, e.g., Youngman et al., 1983). Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

International PCT Publication No. WO2003/083125 discloses methods for modifying Bacillus cells, such as the creation of Bacillus deletion strains and DNA constructs using PCR fusion to bypass E. coli. PCT Publication No. WO2002/14490 discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion.

Those of skill in the art are well aware of suitable methods for introducing polynucleotide sequences into bacterial cells (e.g., E. coli and Bacillus sp.) (e.g., Ferrari et al., 1989; Saunders et al., 1984; Hoch et al., 1967; Mann et al., 1986; Holubova, 1985; Chang et al., 1979; Vorobjeva et al., 1980; Smith et al., 1986; Fisher et al., 1981 and McDonald, 1984). Indeed, such methods as transformation including protoplast transformation and congression, transduction, and protoplast fusion are known and suited for use in the present disclosure. Methods of transformation are particularly preferred to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Bacillus strain by methods known in the art (e.g., Stahl et al., 1984; Palmeros et al., 2000). In some embodiments, resolution of the vector from a host chromosome leaves the flanking regions in the chromosome, while removing the indigenous chromosomal region.

Promoters and promoter sequence regions for use in the expression of genes, open reading frames (ORFs) thereof and/or variant sequences thereof in Bacillus cells are generally known on one of skill in the art. Promoter sequences of the disclosure are generally chosen so that they are functional in the Bacillus cells. Certain exemplary Bacillus promoter sequences include, but are not limited to, the B. subtilis alkaline protease (aprE) promoter, the α-amylase promoter of B. subtilis, the α-amylase promoter of B. amyloliquefaciens, the neutral protease (nprE) promoter from B. subtilis, a mutant aprE promoter (e.g., PCT Publication No. WO2001/51643) or any other promoter from B licheniformis or other related Bacilli.

Methods for screening and creating promoter libraries with a range of activities (promoter strength) in Bacillus cells is describe in PCT Publication No. WO2003/089604.

V. CULTURING MODIFIED CELLS FOR PRODUCTION OF A PROTEIN OF INTEREST

As generally described above, certain embodiments are related to compositions and methods for constructing and obtaining Bacillus cells/strains having increased protein production phenotypes. Thus, certain embodiments are related to methods of producing proteins of interest in Bacillus cells by fermenting/cultivating the cells in a suitable medium. Fermentation methods well known in the art can be applied to ferment the parental and modified (daughter) Bacillus cells of the disclosure.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Bacillus cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

VI. PROTEINS OF INTEREST

A protein of interest (POI) of the instant disclosure can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest. For example, in certain embodiments, a modified Bacillus cell of the disclosure produces at least about 0.10% more, at least about 0.5% more, at least about 1% more, at least about 5% more, at least about 6% more, at least about 7% more, at least about 8% more, at least about 9% more, or at least about 10% or more of a POI, relative to its unmodified (parental) cell.

In certain embodiments, a modified Bacillus cell of the disclosure exhibits an increased specific productivity (Qp) of a POI relative the (unmodified) parental cell. For example, the detection of specific productivity (Qp) is a suitable method for evaluating protein production. The specific productivity (Qp) can be determined using the following equation:

“Qp=gP/gDCW·hr”

wherein, “gP” is grams of protein produced in the tank; “gDCW” is grams of dry cell weight (DCW) in the tank and “hr” is fermentation time in hours from the time of inoculation, which includes the time of production as well as growth time.

Thus, in certain other embodiments, a modified Bacillus cell of the disclosure comprises a specific productivity (Qp) increase of at least about 0.1%, at least about 1%, at least about 5%, at least about 6%, at least about 7%, at least about 8%, at least about 9%, or at least about 10% or more, relative to the unmodified (parental) cell.

In certain embodiments, a POI or a variant POI thereof is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

Thus, in certain embodiments, a POI or a variant POI thereof is an enzyme selected from Enzyme Commission (EC) Number EC 1, EC 2, EC 3, EC 4, EC 5 or EC 6.

In certain other embodiments, a modified Bacillus cell of the disclosure comprises an expression construct encoding an amylase. A wide variety of amylase enzymes and variants thereof are known to one skilled in the art. For example, International PCT Publication NO. WO2006/037484 and WO 2006/037483 describe variant α-amylases having improved solvent stability, PCT Publication No. WO1994/18314 discloses oxidatively stable α-amylase variants, PCT Publication No. WO1999/19467, WO2000/29560 and WO2000/60059 disclose Termamyl-like α-amylase variants, PCT Publication No. WO2008/112459 discloses α-amylase variants derived from Bacillus sp. number 707, PCT Publication No. WO1999/43794 discloses maltogenic α-amylase variants, PCT Publication No. WO1990/11352 discloses hyper-thermostable α-amylase variants, PCT Publication No. WO2006/089107 discloses α-amylase variants having granular starch hydrolyzing activity, and the like.

There are various assays known to those of ordinary skill in the art for detecting and measuring activity of intracellularly and extracellularly expressed proteins.

PCT Publication No. WO2014/164777 discloses Ceralpha α-amylase activity assays useful for detecting amylase activities described herein.

### EXAMPLES

Certain aspects of the present invention may be further understood in light of the following examples, which should not be construed as limiting. Modifications to materials and methods will be apparent to those skilled in the art.

### Example 1

Construction of Cas9 Vectors Targeting Rghr Locus

The Cas9 protein from S. pyogenes (SEQ ID NO: 1) was codon optimized for Bacillus (SEQ ID NO: 2) with the addition of an N-terminal nuclear localization sequence (NLS; “APKKKRKV”; SEQ ID NO: 3), a C-terminal NLS (“KKKKLK”; SEQ ID NO: 4), a deca-histidine tag (“HHHHHHHHHHH”; SEQ ID NO: 5), an aprE promoter sequence from B. subtilis (SEQ ID NO: 6) and a terminator sequence (SEQ ID NO: 7), and was amplified using Q5 DNA polymerase (NEB)per manufacturer's instructions with the forward (SEQ ID NO: 8) and reverse (SEQ ID NO: 9) primer pair set forth below in TABLE 1.

TABLE 1
FORWARD AND REVERSE PRIMER PAIR

| | | |
|---|---|---|
| Forward | ATATATGAGTAAACTTGGTCTGACA | SEQ ID NO: 8 |
| | GAATTCCTCCATTTTCTTCTGCTAT | |
| Reverse | TGCGGCCGCGAATTCGATTACGAAT | SEQ ID NO: 9 |
| | GCCGTCTCCC | |

The backbone (SEQ ID NO: 10) of plasmid pKB320 (SEQ ID NO: 11) was amplified using Q5 DNA polymerase (NEB) per manufacturer's instructions with the forward (SEQ ID NO: 12) and reverse (SEQ ID NO: 13) primer pair set forth below in TABLE 2.

TABLE 2
FORWARD AND REVERSE PRIMER PAIR

| | | |
|---|---|---|
| Forward | GGGAGACGGCATTCGTAATCGAATT | SEQ ID NO: 12 |
| | CGCGGCCGCA | |
| Reverse | ATAGCAGAAGAAAATGGAGGAATTC | SEQ ID NO: 13 |
| | TGTCAGACCAAGTTTACTCATATAT | |

The PCR products were purified using Zymo clean and concentrate 5 columns per manufacturer's instructions. Subsequently, the PCR products were assembled using prolonged overlap extension PCR (POE-PCR) with Q5 Polymerase (NEB) mixing the two (2) fragments at equimolar ratio. The POE-PCR reactions were cycled as follows: 98° C. for five (5) seconds, 64° C. for ten (10) seconds and 72° C. for four (4) minutes and (15) fifteen seconds for 30 cycles. Five (5) μl of the POE-PCR (DNA) was transformed into Top10 E. coli (Invitrogen) per manufacturer's instructions and selected on lysogeny (L) Broth (Miller recipe; 1% (w/v) Tryptone, 0.5% Yeast extract (w/v), 1% NaCl (w/v)), containing fifty (50) μg/ml kanamycin sulfate and solidified with 1.5% Agar. Colonies were allowed to grow for eighteen (18) hours at 37° C. Colonies were picked and plasmid DNA prepared using Qiaprep DNA miniprep kit per manufacturer's instructions and eluted in fifty-five (55) μl of ddH₂0. The plasmid DNA was Sanger sequenced to verify correct assembly, using the sequencing primers set forth below in TABLE 3.

TABLE 3
SEQUENCING PRIMERS

| | | |
|---|---|---|
| Reverse | CCGACTGGAGCTCCTATATTACC | SEQ ID NO: 14 |
| Reverse | GCTGTGGCGATCTGTATTCC | SEQ ID NO: 15 |
| Forward | GTCTTTTAAGTAAGTCTACTCT | SEQ ID NO: 16 |
| Forward | CCAAAGCGATTTTAAGCGCG | SEQ ID NO: 17 |
| Forward | CCTGGCACGTGGTAATTCTC | SEQ ID NO: 18 |
| Forward | GGATTTCCTCAAATCTGACG | SEQ ID NO: 19 |
| Forward | GTAGAAACGCGCCAAATTACG | SEQ ID NO: 20 |
| Forward | GCTGGTGGTTGCTAAAGTCG | SEQ ID NO: 21 |
| Forward | GGACGCAACCCTCATTCATC | SEQ ID NO: 22 |
| Reverse | CAGGCATCCGATTTGCAAGG | SEQ ID NO: 23 |
| Forward | GCAAGCAGCAGATTACGCG | SEQ ID NO: 24 |

The correctly assembled plasmid, pRF694 (SEQ ID NO: 25) was used to construct plasmids pRF801 (SEQ ID NO: 26) and pRF806 (SEQ ID NO: 27) for editing the B. licheniformis genome at target site 1 (TS1; SEQ ID NO: 28) and target site 2 (TS2; SEQ ID NO: 29) as described below.

The serA1 open reading frame (SEQ ID NO: 30) of B. licheniformis contains a unique target site (TS), target site 1 (TS1; SEQ ID NO: 28) in the reverse orientation. The TS1 lies adjacent to a proto-spacer adjacent motif (PAM; SEQ ID NO: 31) in the reverse orientation. The target site can be converted into the DNA encoding a variable targeting (VT) domain (SEQ ID NO: 32). The DNA sequence encoding the VT domain (SEQ ID NO: 32) is operably fused to the DNA sequence encoding the Cas9 endonuclease recognition domain (CER, SEQ ID NO: 33), such that when transcribed by RNA polymerase of the bacterial cell, it produces a functional guide RNA (gRNA) targeting target site 1 (SEQ ID NO: 34). The DNA encoding the gRNA was operably linked to a promoter operable in Bacillus sp. cells (e.g., the spac promoter; SEQ ID NO: 35) and a terminator sequence operable in Bacillus sp. cells (e.g., the t0 terminator sequence of phage lambda; SEQ ID NO: 36), such that the promoter was positioned 5′ of the DNA encoding the gRNA (SEQ ID NO: 33) and the terminator is positioned 3′ of the DNA encoding the gRNA (SEQ ID NO: 33).

An editing template to delete the serA1 gene in response to Cas9/gRNA cleavage was created by amplification of two homology arms from B. licheniformis genomic DNA (gDNA). The first fragment (homology arm 1) corresponds to the five hundred (500) nucleotides directly upstream (5′) of the serA1 ORF (SEQ ID NO: 37). This fragment was amplified using Q5 DNA polymerase per the manufacturer's instructions and the forward (SEQ ID NO: 38) and reverse (SEQ ID NO: 39) primers listed below in TABLE 4. The primers incorporate eighteen (18) nucleotides homologous to the 5′ end of the second fragment on the 3′ end of the first fragment, and twenty (20) nucleotides homologous to pRF694 to the 5′ end of first fragment.

TABLE 4
FORWARD AND REVERSE PRIMER PAIR

| | | |
|---|---|---|
| Forward | TGAGTAAACTTGGTCTGACAAAT | SEQ ID NO: 38 |
| | GGTTCTTTCCCCTGTCC | |
| Reverse | AGGTTCCGCAGCTTCTGTGTAAG | SEQ ID NO: 39 |
| | ATTTCCTCCTAAATAAGCGTCAT | |

The second fragment (homology arm 2) corresponds to the five-hundred (500) nucleotides directly downstream of the 3′ end of the serA1 ORF (SEQ ID NO: 40). This fragment was amplified using Q5 DNA polymerase per manufacturer's instructions and the forward (SEQ ID NO: 41) and reverse (SEQ ID NO: 42) primers listed below in TABLE 5. The primers incorporate twenty-eight (28) nucleotides homologous to the 3′ end of the first fragment on the 5′ end of the second fragment and twenty-one (21) nucleotides homologous to pRF694 on the 3′ end of the second fragment. PGP25,DNA

TABLE 5
FORWARD AND REVERSE PRIMER PAIR

| | | |
|---|---|---|
| Forward | ATGACGCTTATTTAGGAGGAAATCTTACACAGAA | SEQ ID |
| | GCTGCGGAACCT | NO: 41 |
| Reverse | CAGAAGAAAATGGAGGAATTCGAATATCGACCGG | SEQ ID |
| | AACCCAC | NO: 42 |

The DNA encoding the target site 1 gRNA expression cassette (SEQ ID NO: 43), the first homology arm (SEQ ID NO: 37) and second homology arm (SEQ ID NO: 40) were assembled into pRF694 (SEQ ID NO: 25) using standard molecular biology techniques, generating plasmid pRF801 (SEQ ID NO: 26), an E. coli-B. licheniformis shuttle plasmid containing a Cas9 expression cassette (SEQ ID NO: 2), a gRNA expression cassette (SEQ ID NO: 43) encoding a gRNA targeting TS1 within the serA1 ORF and an editing template (SEQ ID NO: 44) composed of the first homology arm (SEQ ID NO: 37) and second homology arm (SEQ ID NO: 40). The plasmid was verified by Sanger sequencing using the oligonucleotides (primers) set forth above in TABLE 3.

The rghR1 open reading frame of B. licheniformis (SEQ ID NO: 45) contains a unique target site (TS) on the reverse strand, target site 2 (TS2; SEQ ID NO: 28). The target site lies adjacent to a proto-spacer adjacent motif (PAM; SEQ ID NO: 46) on the reverse strand. The target site can be converted into the DNA encoding a variable targeting (VT) domain (SEQ ID NO: 47). The DNA sequence encoding the VT domain (SEQ ID NO: 47) is operably fused to the DNA sequence encoding the Cas9 endonuclease recognition domain (CER; SEQ ID NO: 33), such that when transcribed by RNA polymerase of the bacterial cell, it produces a functional gRNA targeting target site 2 (SEQ ID NO: 48). The DNA encoding the gRNA was operably linked to a promoter operable in Bacillus sp. cells (e.g., the spac promoter from B. subtilis; SEQ ID NO: 35) and a terminator operable in Bacillus sp. cells (e.g., the t0 terminator of phage lambda; SEQ ID NO: 36), such that the promoter was positioned 5′ of the DNA encoding the gRNA (SEQ ID NO: 48) and the terminator is positioned 3′ of the DNA encoding the gRNA (SEQ ID NO: 48).

An editing template to modify the rghR1 gene in response to Cas9/gRNA cleavage was created by amplification of two homology arms from B. licheniformis genomic DNA (gDNA). The first fragment corresponds to the 500 nucleotides directly upstream (5′) of the rghR1 ORF (homology arm 1; SEQ ID NO: 49). This fragment was amplified using Q5 DNA polymerase per the manufacturer's instructions and the forward (SEQ ID NO: 50) and reverse (SEQ ID NO: 51) primers listed below in TABLE 6. The primers incorporate twenty-three (23) nucleotides homologous to the 5′ end of the second fragment on the 3′ end of the first fragment and twenty (20) nucleotides homologous to pRF694 to the 5′ end of first fragment.

TABLE 6
FORWARD AND REVERSE PRIMER PAIR

| | | |
|---|---|---|
| Forward | TGAGTAAACTTGGTCTGACATTGATATTCAGCAC | SEQ ID |
| | CCTGCG | NO: 50 |
| Reverse | TGTGCCGCGGAGAAGTATGGCCAAAACCTCGCAA | SEQ ID |
| | TCTC | NO: 51 |

The second fragment corresponds to the 500 nucleotides directly downstream of the 3′ end of the rghR1 ORF (homology arm 2; SEQ ID NO: 52). This fragment was amplified using Q5 DNA polymerase per manufacturer's instructions and the forward (SEQ ID NO: 53) and reverse (SEQ ID NO: 54) primers listed below in TABLE 7. The primers incorporate twenty (20) nucleotides homologous to the 3′ end of the first fragment on the 5′ end of the second fragment and twenty-one (21) nucleotides homologous to pRF694 on the 3′ end of the second fragment.

TABLE 7
FORWARD AND REVERSE PRIMER PAIR

| | | |
|---|---|---|
| Forward | GAGATTGCGAGGTTTTGGCCATACTTCTCCGCGG | SEQ ID |
| | CACA | NO: 53 |
| Reverse | CAGAAGAAAATGGAGGAATTCATTTCTCGGGTTT | SEQ ID |
| | AAACAGCCAC | NO: 54 |

The DNA encoding the target site 2 gRNA expression cassette (SEQ ID NO: 55), the first homology arm (SEQ ID NO: 49) and second homology arm (SEQ ID NO: 52) were assembled into pRF694 (SEQ ID NO: 25) using standard molecular biology techniques, generating pRF806 (SEQ ID NO: 27), an E. coli-B. licheniformis shuttle plasmid containing a Cas9 expression cassette (SEQ ID NO: 2), a gRNA expression cassette (SEQ ID NO:55) encoding a gRNA targeting target site 2 within the rghR1 ORF, and an editing template (SEQ ID NO: 56) composed of the first homology arm (SEQ ID NO: 49) and second homology arm (SEQ ID NO: 52). The plasmid was verified by Sanger sequencing with the oligonucleotides (primers) set forth above in TABLE 3.

### Example 2

Construction of Cas9 Y155H Variant and Associated Targeting Plasmids

In the present example, the Y155H variant of S. pyogenes Cas9 (SEQ ID NO:57) was constructed in the pRF801 (SEQ ID NO: 26) and pRF806 plasmids (SEQ ID NO: 27). To introduce the (Cas9) Y155H variant in the pRF801 plasmid (SEQ ID NO: 26) or the pRF806 plasmid (SEQ ID NO: 27), site-directed mutagenesis was performed using Quikchange mutagenesis kit per the manufacturer's instructions and the forward (SEQ ID NO: 58) and reverse (SEQ ID NO: 59) primers presented below in TABLE 8, using pRF801 plasmid (SEQ ID NO: 26) or pRF806 plasmid (SEQ ID NO: 27) as template DNA.

TABLE 8
FORWARD AND REVERSE PRIMER PAIR

| | | |
|---|---|---|
| Forward | GATCTGCGTTTAATCCATCTTGCGTTAGCGCAC | SEQ ID |
| | | NO: 58 |
| Reverse | GTGCGCTAACGCAAGATGGATTAAACGCAGATC | SEQ ID |
| | | NO: 59 |

The resultant products of the reaction, pRF827 (SEQ ID NO: 60) comprised a (Cas9) Y155H variant expression cassette (SEQ ID NO: 61), a gRNA expression cassette (SEQ ID NO: 43) encoding a gRNA targeting site 1 (TS1) within the serA1 ORF, and an editing template (SEQ ID NO: 44) composed of the first (SEQ ID NO: 37) and second (SEQ ID NO: 40) homology arms; or pRF856 (SEQ ID NO: 62) which comprised a (Cas9) Y155H variant expression cassette (SEQ ID NO: 61), a gRNA expression cassette (SEQ ID NO: 55) targeting site 2 (TS2) within the rghR1 ORF and an editing template (SEQ ID NO: 56) composed of the first (SEQ ID NO: 49) and second (SEQ ID NO: 52) homology arms. The plasmid DNAs were Sanger sequenced to verify correct assembly, using the sequencing oligonucleotides (primers) set forth above in TABLE 3.

Construction of Plasmid pRF862

Plasmid pRF862 (SEQ ID NO: 77) was constructed by moving a fragment (SEQ ID NO: 63) of the Cas9 ORF comprising the Y155H (variant) substitution from pRF827 (SEQ ID NO: 60) and amplified using the forward (SEQ ID NO: 64) and reverse (SEQ ID NO: 65) primers presented below in TABLE 9.

TABLE 9
FORWARD AND REVERSE PRIMER PAIR

| | | |
|---|---|---|
| Forward | CACGTCGTAAAAATCGTATT | SEQ ID NO: 64 |
| Reverse | CAAACAGACCATTTTTCTTT | SEQ ID NO: 65 |

A second fragment (SEQ ID NO: 67) was amplified from pRF694 (SEQ ID NO: 66) such that it comprised the entire plasmid, except the fragment contained on the pRF827 fragment above (SEQ ID NO: 60). This fragment shares homology with the 5′ and 3′ ends of the pRF827 fragment (SEQ ID NO: 60) for assembly, and was amplified using the forward (SEQ ID NO: 68) and reverse (SEQ ID NO: 69) primers set forth below in TABLE 10.

TABLE 10
FORWARD AND REVERSE PRIMER PAIR

| | | |
|---|---|---|
| Forward | AAAGAAAAATGGTCTGTTTG | SEQ ID NO: 68 |
| Reverse | AATACGATTTTTACGACGTG | SEQ ID NO: 69 |

The two (2) fragments were assembled using NEBuilder according to manufacturer's instructions and transformed into E. coli competent cells. Plasmid sequence was verified by the method of Sanger using the oligonucleotides (primers) as set forth above in TABLE 3. A sequence verified isolate was stored as plasmid pRF862 (SEQ ID NO:77).

pRF869 (SEQ ID NO: 70), a plasmid that targets the rghR2 ORF (SEQ ID NO: 71) and inserts three (3) in-frame stop codons, was constructed using two (2) parts. The first part (SEQ ID NO: 72) comprising the editing template (SEQ ID NO: 73) to modify the rghR2 ORF (SEQ ID NO: 71), and a gRNA expression cassette (SEQ ID NO: 74) targeting the rghR2 ORF (SEQ ID NO: 71) was synthesized by IDT and was amplified for assembly using the forward (SEQ ID NO: 75) and reverse (SEQ ID NO: 76) primers set forth below in TABLE 11.

TABLE 11
FORWARD AND REVERSE PRIMER PAIR

| | | |
|---|---|---|
| Forward | CGTGCGGCCGCGAATTC | SEQ ID NO: 75 |
| Reverse | CCTGATACCGGGAGACGGCATTCGTAATC | SEQ ID NO: 76 |

A second part (SEQ ID NO: 77) from pRF862 (SEQ ID NO: 77), comprising the Cas9 expression cassette and all plasmid components were amplified using the forward (SEQ ID NO: 78) and reverse (SEQ ID NO: 79) primers set forth below in TABLE 12.

TABLE 12
FORWARD AND REVERSE PRIMER PAIR

| | | |
|---|---|---|
| Forward | GAATTCGCGGCCGCACG | SEQ ID NO: 78 |
| Reverse | GATTACGAATGCCGTCTCCCGGTATCAGG | SEQ ID NO: 79 |

The two parts were assembled using NEBuilder according to manufacturer's instructions and transformed into E. coli. Plasmid sequence was verified by the method of Sanger using the oligonucleotides (primers) set forth above in TABLE 3. A sequence verified isolate was stored as pRF869 (SEQ ID NO: 70).

Several additional Cas9 plasmids were assembled as described above in Examples 1 and 2. Those plasmids are listed below in TABLE 13, along with the target site (TS) sequence and the editing template effect. As used below in TABLE 13, the term “SID” is an abbreviation for “SEQ ID” number.

TABLE 13
ADDITIONAL CAS9 PLASMIDS FOR EDITING B. LICHENIFORMIS CELLS

| | | | | Editing | Editing |
|---|---|---|---|---|---|
| | | | Target | Template | Template |
| Plasmid | SID | TS and PAM Sequence | SID | Effect | SID |
| pRF874 | 80 | GATGCCATCAGTTCCTCATACGG | 81 | ΔrghR1 | 82 |
| pRF879 | 83 | GCGAGCGGCTCAAAGAGCTGAGG | 84 | ΔrghR2 | 85 |
| pRF899 | 86 | GATGTATTCCGGCGTCAGTTCGG | 87 | ΔrghR2 | 88 |
| | | | | ΔrghR1 | |
| pRF901 | 89 | GATGTATTCCGGCGTCAGTTCGG | 87 | ΔrghR2 | 90 |
| | | | | ΔrghR1 | |
| | | | | ΔBli3644 | |
| | | | | ΔyvzC | |

### Example 3

Construction of Amylase Expressing Bacillus Strains Comprising Various Rghr Locus Alleles

In the present example, a series of rghR locus alleles were introduced into a parental B. licheniformis strain comprising an expression cassette encoding a variant Cytophaga sp. α-amylase (e.g., a variant Cytophaga sp. α-amylase described in PCT Publication No. WO2017/100720, incorporated herein by reference in its entirety). More particularly, the parental B. licheniformis strain, named LDN143, comprises (a) a native rghR locus, (b) a deletion of the serA gene (SEQ ID NO: 30), a deletion of the lysA genes (SEQ ID NO: 92), and two (2) α-amylase expression cassettes.

For example, the first expression cassette (SEQ ID NO: 93), integrated in the serA locus, comprises a serA ORF (SEQ ID NO: 30) and the synthetic p3 promoter (SEQ ID NO: 94; described in PCT Publication No. WO2017/152169) operably linked to the DNA encoding the B. subtilis aprE 5′-UTR (SEQ ID NO: 95) operably linked to the DNA encoding B. licheniformis amyL signal sequence (SEQ ID NO: 96) operably linked to the DNA sequence encoding the Cytophaga sp. variant alpha amylase (SEQ ID NO: 97) operably linked to the B licheniformis amyL transcriptional terminator (SEQ ID NO: 98). The second expression cassette (SEQ ID NO: 99), integrated in the amyL locus, comprises the lysA auxotrophic marker (SEQ ID NO: 92) and the B. licheniformis amyL promoter (SEQ ID NO: 100) operably linked to the DNA encoding B. subtilis aprE 5′-UTR (SEQ ID NO: 95) operably linked to the DNA encoding the amyL signal sequence (SEQ ID NO: 96) operably linked to the DNA sequence encoding the Cytophaga sp. variant alpha amylase (SEQ ID NO: 97) operably linked to the B licheniformis amyL transcriptional terminator (SEQ ID NO: 98).

A version of the LDN143 cell/strain comprising the pB1.comK plasmid (SEQ ID NO: 101), which contains a spectinomycin marker (SEQ ID NO: 102), the DNA encoding the Xy1R repressor (SEQ ID NO: 103) and the xy1A promoter (SEQ ID NO: 104) operably linked to the DNA encoding the B. licheniformis ComK protein (SEQ ID NO: 105) (e.g., see Liu and Zuber, 1998; Hamoen et al., 1998; US Patent Publication No. 2006/0199222) was transformed with pRF869 (SEQ ID NO: 70), pRF874 (SEQ ID NO: 80), pRF879 (SEQ ID NO: 83), pRF899 (SEQ ID NO: 86), or pRF901 (SEQ ID NO: 89) plasmids amplified using rolling circle amplification (TruePrime RCA, Lucigen).

Briefly, the LDN143/pBl.comK competent cells were generated. The LDN143/pBl.comK strain was grown overnight in L broth containing one hundred (100) ppm spectinomycin at 37° C. and 250 RPM shaking. The culture was diluted to an OD₆₀₀ of 0.7 in fresh L broth containing one hundred (100) ppm spectinomycin. This new culture was grown for one (1) hour at 37° C. and 250RPM. D-xylose was added to 0.1% w v⁻¹ and the culture was grown for an additional four (4) hours. The cells were harvest at 1700 g for seven (7) minutes. The cells were resuspended in one-fourth (¼%) culture volume of spent medium containing 10% v·v⁻¹ DMSO. One hundred (100) μl of cells were mixed with ten (10) μl of pRF869 (SEQ ID NO: 70), pRF874 (SEQ ID NO: 80), pRF879 (SEQ ID NO: 83), pRF899 (SEQ ID NO: 86), or pRF901 (SEQ ID NO: 89) plasmid RCA amplification product. The cell/DNA mixture was incubated at 37° C. 1400 RPM for one and a half (1.5) hours. The mixture was then plated onto L agar plates containing twenty (20) ppm kanamycin. The inoculated plates were incubated at 37° C. for forty-eight to seventy-two (48-72) hours. Colonies that formed on L agar containing twenty (20) ppm kanamycin were screened using colony PCR to confirm modification of the locus as described below.

For cells transformed with pRF869 (SEQ ID NO: 70), the rghR2 gene was amplified using standard PCR techniques using the forward (SEQ ID NO: 106) and reverse (SEQ ID NO: 107) primers listed below in TABLE 14.

TABLE 14
FORWARD AND REVERSE PRIMER PAIR

| | | |
|---|---|---|
| Forward | GCGAATCGAAAACGGAAAGC | SEQ ID NO: 106 |
| Reverse | TCATCGCGATCGGCATTACG | SEQ ID NO: 107 |

This PCR product is a 1,164 nucleotide fragment comprising the targeted region of rghR2 (SEQ ID NO: 108) was sequenced using the method of Sanger to confirm the introduction of the rghR2ₛₜₒₚ allele (SEQ ID NO: 109), comprising three (3) in-frame nonsense mutations using the forward (SEQ ID NO: 110) primer set forth below in TABLE 15. An isolate with the rghR2ₛₜₒₚ allele (SEQ ID NO: 109) was stored as strain BF314.

TABLE 15
RGHR2_{STOP} SEQUENCING PRIMER

| | | |
|---|---|---|
| Forward | TTTCGACTTTCTCGTGCAGG | SEQ ID NO: 110 |

For cells transformed with pRF874 (SEQ ID NO: 80), the rghR1 gene region was amplified using the forward (SEQ ID NO: 111) and reverse (SEQ ID NO: 112) primers set forth below in TABLE 16.

TABLE 16
FORWARD AND REVERSE PRIMER PAIR

| | | |
|---|---|---|
| Forward | ATCAAACATGCCATGTTTGC | SEQ ID NO: 111 |
| Reverse | AGGTTGAGCAGGTCTTCG | SEQ ID NO: 112 |

The native rghR1 fragment (SEQ ID NO: 113) produced by the primers in TABLE 16 is 1,499 nucleotides in length. When the rghR1 gene is deleted (ΔrghR1), the fragment (SEQ ID NO: 114) produced by the primers in TABLE 16 is 1,097 nucleotides in length, and is visibly smaller upon electrophoresis.

An isolate of LDN143 comprising the deleted rghR1 allele (ΔrghR1; SEQ ID NO: 114) was stored as strain BF324.

For cells transformed with pRF879 (SEQ ID NO: 83), the rghR2 gene locus was amplified using the forward (SEQ ID NO: 115) and reverse (SEQ ID NO: 116) primers set forth below in TABLE 17.

TABLE 17
FORWARD AND REVERSE PRIMER PAIR

| | | |
|---|---|---|
| Forward | GAGATTGCGAGGTTTTGGCC | SEQ ID NO: 115 |
| Reverse | GGCATACGGCGTATTGTTCG | SEQ ID NO: 116 |

The native rghR2 fragment (SEQ ID NO: 117) produced by the primers in TABLE 17 is 1,629 nucleotides in length. When the rghR2 gene is deleted (ΔrghR2), the fragment (SEQ ID NO: 118) produced by the primers in TABLE 17 is 1,248 nucleotides in length, and is visibly smaller upon electrophoresis. An isolate of LDN143 comprising the ΔrghR2 locus allele (SEQ ID NO: 118) was stored as strain BF377.

For cells transformed with pRF899 (SEQ ID NO: 86), the rghR2 rghR1 region was amplified using the forward (SEQ ID NO: 119) and reverse (SEQ ID NO: 120) primers set forth below in TABLE 18.

TABLE 18
FORWARD AND REVERSE PRIMER PAIR

| | | |
|---|---|---|
| Forward | ATGATATTTTCGCCGTCGGT | SEQ ID NO: 119 |
| Reverse | AACGATGCAGGAGCTCAATT | SEQ ID NO: 120 |

The native rghR2 rghR1 fragment (SEQ ID NO: 121) produced by primers in TABLE 18 from parent strain LDN143 was 2,353 nucleotides in length. When the rghR2 and rghR1 genes are deleted (ΔrghR2 ΔrghR1), the fragment (SEQ ID NO: 122) produced by the primers in TABLE 18 is 1,401 nucleotides in length, and is visibly smaller upon electrophoresis. An isolate of LDN143 comprising the ΔrghR2 ΔrghR1 allele (SEQ ID NO: 122) was stored as BF389.

For cells transformed with pRF901 (SEQ ID NO: 89), the rghR2 locus was amplified using the forward (SEQ ID NO: 123) and reverse (SEQ ID NO: 124) primers set forth below in TABLE 19.

TABLE 19
FORWARD AND REVERSE PRIMER PAIR

| | | |
|---|---|---|
| Forward | CATGACGTCTTTCCACCAGT | SEQ ID NO: 123 |
| Reverse | AACGATGCAGGAGCTCAATT | SEQ ID NO: 124 |

The native rghR2 fragment (SEQ ID NO: 125) produced by primers in TABLE 19 from parent strain LDN143 was 3,265 nucleotides in length. When the rghR2, rghR1, yvzC and 3644 genes are deleted (ΔrghR2, ΔrghR1, ΔyvzC and A3644), the fragment (SEQ ID NO: 126) produced by the primers in TABLE 19 is 1,596 nucleotides in length, and is visibly smaller upon electrophoresis. An isolate of LDN143 comprising the ΔrghR2, ΔrghR1, ΔyvzC and A3644 alleles was stored as BF391.

### Example 4

Amylase Production in Bacillus Strains with a Modified Rghr Locus

In order to determine the effects of the various rghR locus alleles on the production of an α-amylase, the strains were grown under standard small-scale assay conditions in triplicate, as generally described in PCT Publication No. WO2018/156705 (incorporated herein by reference in its entirety). The yield of the variant (Cytophaga sp.) α-amylase was determined by using Bradford protein assay (Peirce) per manufacturer's instructions. Thus, the average α-amylase production for each strain was determined and normalized to the parent strain LDN143, as shown below in TABLE 20.

TABLE 20
RELATIVE YIELD OF AMYLASE PRODUCTION
FOR DIFFERENT RGHR LOCUS ALLELES

| | Relative | rghR locus | Relative |
|---|---|---|---|
| Strain | Genotype | SEQ ID | yield ± SEM |
| LDN143 | | SEQ ID NO: 127 | 1.00 ± 0.10 |
| BF314 | rghR2ₛₜₒₚ | SEQ ID NO: 128 | 1.23 ± 0.07 |
| BF324 | ΔrghR1 | SEQ ID NO: 129 | 1.26 ± 0.13 |
| BF377 | ΔrghR2 | SEQ ID NO: 130 | 1.62 ± 0.08 |
| BF389 | ΔrghR2 ΔrghR1 | SEQ ID NO: 131 | 1.35 ± 0.02 |
| BF391 | ΔrghR2 ΔrghR1 | SEQ ID NO: 132 | 1.30 ± 0.02 |
| | ΔyvzC Δ3644 | | |

As presented above in TABLE 20, the B. licheniformis cells/strains with mutations in the rghR locus demonstrate increased production of the heterologous α-amylase protein, with approximately 23-62% more amylase protein produced than the comparable parental cell (LDN143), that is wild-type for the rghR locus.

### Example 5

Pulcherrimin Production in Bacillus Strains with a Modified Rghr Locus

As briefly stated above in section III, a particular feature of the rghR locus is the transcriptional control of the operon responsible for producing the iron scavenging pigment pulcherriminic acid. For example, pulcherriminic acid is known to react with ferric iron outside the cell to form an insoluble red pigment. This red pigment can be re-solubilized as the sodium salt and quantified using absorbance at 410 nm (Uffen and Canale-Parola, 1972). Briefly ten (10) ml of culture supernatant was harvested at 4000 RPM for ten (10) minutes. The pellet was washed 2× with water. The pellet was resuspended in one (1) ml of 1N NaOH and incubated at room temperature for ten (10) minutes to allow the conversion of the insoluble pulcherrimin to the soluble sodium pulcherrimate. The remaining debris was removed with a brief centrifuge at 14000 RPM. The absorbance at 410 nm was measured against a 1N NaOH blank.

TABLE 21
QUANTIFICATION OF PULCHERRIMINIC ACID

| Strain | Relative Genotype | rghR2 locus SEQ ID NO | Relative A₄₁₀ |
|---|---|---|---|
| LDN143 | | SEQ ID NO: 127 | 1.0 |
| BF314 | rghR2ₛₜₒₚ | SEQ ID NO: 128 | 0.7 |
| BF324 | ΔrghR1 | SEQ ID NO: 129 | 1.1 |
| BF377 | ΔrghR2 | SEQ ID NO: 130 | 0.5 |
| BF389 | ΔrghR2 ΔrghR1 | SEQ ID NO: 131 | 1.2 |
| BF391 | ΔrghR2 ΔrghR1 | SEQ ID NO: 132 | 1.1 |
| | ΔyvzC Δ3644 | | |

Thus, as shown above in TABLE 21, several mutations in the rghR locus significantly decreased the production of pulcherrimin to about 30-50% (e.g., BF314 and BF377) relative to the parent, while several other mutations increased the production of pulcherrimin to about 10-20% (e.g., BF324, BF389 and BF391) relative to the parent, indicating that mutations in the rghR locus control the biosynthesis of pulcherriminic acid.

To measure the relative yield of biomass for the various strains while producing the heterologous amylase protein, the optical density (OD) of two-hundred (200) μl of culture was measured at 600 nm, as presented below in TABLE 22.

TABLE 22
RELATIVE OPTICAL DENSITY

| Strain | Relative Genotype | rghR2 locus SEQ ID NO | Relative OD₆₀₀ |
|---|---|---|---|
| LDN143 | | SEQ ID NO: 127 | 1.00 ± 0.08 |
| BF314 | rghR2ₛₜₒₚ | SEQ ID NO: 128 | 1.10 ± 0.09 |
| BF324 | ΔrghR1 | SEQ ID NO: 129 | 1.03 ± 0.16 |
| BF377 | ΔrghR2 | SEQ ID NO: 130 | 1.15 ± 0.12 |
| BF389 | ΔrghR2 ΔrghR1 | SEQ ID NO: 131 | 1.09 ± 0.03 |
| BF391 | ΔrghR2 ΔrghR1 | SEQ ID NO: 132 | 1.09 ± 0.09 |
| | ΔyvzC Δ3644 | | |

### REFERENCES

PCT Publication No. WO1994/18314
PCT Publication No. WO1999/19467
PCT Publication No. WO1999/43794
PCT Publication No. WO2000/29560
PCT Publication No. WO2000/60059
PCT Publication No. WO2004/011609
PCT Publication No. WO2006/037483
PCT Publication No. WO2006/037484
PCT Publication No. WO2006/089107
PCT Publication No. WO2008/112459
PCT Publication No. WO2014/164777
PCT Publication No. WO2018/156705
Albertini and Galizzi, Bacteriol., 162:1203-1211, 1985.
Bergmeyer et al., “Methods of Enzymatic Analysis” vol. 5, Peptidases, Proteinases and their Inhibitors, Verlag Chemie, Weinheim, 1984.
Botstein and Shortle, Science 229: 4719, 1985.
Brode et al., “Subtilisin BPN’ variants: increased hydrolytic activity on surface-bound substrates via decreased surface activity”, Biochemistry, 35(10):3162-3169, 1996.
Caspers et al., “Improvement of Sec-dependent secretion of a heterologous model protein in Bacillus subtilis by saturation mutagenesis of the N-domain of the AmyE signal peptide”, Appl. Microbiol. Biotechnol., 86(6):1877-1885, 2010.

Chang et al., Mol. Gen. Genet., 168:11-115, 1979.

Christianson et al., Anal. Biochem., 223:119-129, 1994.

Devereux et a/., Nucl. Acid Res., 12: 387-395, 1984.

Earl et al., “Ecology and genomics of Bacillus subtilis”, Trends in Microbiology., 16(6):269-275, 2008.
Ferrari et al., “Genetics,” in Harwood et al. (ed.), Bacillus, Plenum Publishing Corp., 1989.
Fisher et. al., Arch. Microbiol., 139:213-217, 1981.
Guerot-Fleury, Gene, 167:335-337, 1995.
Hamoen et al., “Controlling competence in Bacillus subtilis: shared used of regulators”, Microbiology, 149:9-17, 2003.
Hamoen et al., Genes Dev. 12:1539-1550, 1998.
Hampton et al., Seroloaical Methods, A Laboratory Manual, APS Press, St. Paul, Minn., 1990.
Hardwood and Cutting (eds.) Molecular Biological Methods for Bacillus, John Wiley & Sons, 1990.
Hayashi et al., 2006
Hayashi et al., Mol. Microbiol., 59(6): 1714-1729, 2006
Higuchi et al., Nucleic Acids Research 16: 7351, 1988.
Ho et al., Gene 77: 61, 1989.
Hoch et al., J. Bacteriol., 93:1925-1937, 1967.
Holubova, Folia Microbiol., 30:97, 1985.
Hopwood, The Isolation of Mutants in Methods in Microbiology (J. R. Norris and D. W. Ribbons, eds.) pp 363-433, Academic Press, New York, 1970.
Horton et al., Gene 77: 61, 1989.
Hsia et al., Anal Biochem., 242:221-227, 1999.
Iglesias and Trautner, Molecular General Genetics 189: 73-76, 1983.
Jensen et al., “Cell-associated degradation affects the yield of secreted engineered and heterologous proteins in the Bacillus subtilis expression system” Microbiology, 146 (Pt 10:2583-2594, 2000.
Liu and Zuber, 1998,
Lo et al., Proceedings of the National Academy of Sciences USA 81: 2285, 1985.
Maddox et al., J. Exp. Med., 158:1211, 1983.
Mann et al., Current Microbiol., 13:131-135, 1986.
McDonald, J. Gen. Microbiol., 130:203, 1984.
MacDonald, “Biosynthesis of pulcherriminic acid”, Biochem. J, 96: 533-538, 1965.
Needleman and Wunsch, J Mol. Biol., 48: 443, 1970.
Ogura & Fujita, FEMSMicrobiol Lett., 268(1): 73-80. 2007.
Olempska-Beer et al., “Food-processing enzymes from recombinant microorganisms—a review”” Regul.
Toxicol. Pharmacol., 45(2):144-158, 2006.
Palmeros et al., Gene 247:255-264, 2000.
Parish and Stoker, FEMSMicrobiology Letters 154: 151-157, 1997.
Pearson and Lipman, Proc. Natl. Acad. Sci. USA 85: 2444, 1988.
Perego, 1993, In A. L. Sonneshein, J. A. Hoch, and R. Losick, editors, Bacillus subtilis and Other Gram-Positive Bacteria, Chapter 42, American Society ofMicrobiology, Washington, D.C.
Raul et al., “Production and partial purification of alpha amylase from Bacillus subtilis (MTCC 121) using solid state fermentation”, Biochemistry Research International, 2014.
Sarkar and Sommer, BioTechniques 8: 404, 1990.
Saunders et al., J. Bacteriol., 157:718-726, 1984.
Shimada, Meth. Mol. Biol. 57: 157; 1996
Smith and Waterman, Adv. Appl. Math., 2: 482, 1981.
Smith et al., Appl. Env. Microbiol., 51:634 1986.
Stahl and Ferrari, J. Bacteriol., 158:411-418, 1984.
Stahl et al, J. Bacteriol., 158:411-418, 1984.
Tarkinen, et al, J. Biol. Chem. 258: 1007-1013, 1983.
Trieu-Cuot et al., Gene, 23:331-341, 1983.
Uffen and Canale-Parola, “Synthesis of pulcherriminic acid by Bacillus subtilis”, J Bacteriol 111(1): 86-93, 1972.
Van Dijl and Hecker, “Bacillus subtilis: from soil bacterium to super-secreting cell factory”, Microbial Cell Factories, 12(3). 2013.
Vorobjeva et al., FEMSMicrobiol. Lett., 7:261-263, 1980.
Ward, “Proteinases,” in Fogarty (ed.)., Microbial Enzymes and Biotechnology. Applied Science, London, pp 251-317, 1983.
Wells et al., Nucleic Acids Res. 11:7911-7925, 1983.
Westers et al., “Bacillus subtilis as cell factory for pharmaceutical proteins: a biotechnological approach to optimize the host organism”, Biochimica et Biophysica Acta., 1694:299-310, 2004.
Yang et al, J. Bacteriol., 160: 15-21, 1984.
Yang et al., Nucleic Acids Res. 11: 237-249, 1983.
Youngman et al., Proc. Natl. Acad. Sci. USA 80: 2305-2309, 1983.

1. A modified Bacillus licheniformis cell derived from a parental B. licheniformis cell comprising a native rghR chromosomal locus, wherein the modified cell comprises at least one genetic modification of the rghR chromosomal locus selected from the group consisting of (a) a modified rghR1 gene, (b) a modified rghR2 gene, (c) a modified rghR1 gene and modified rghR2 gene, and (d) a modified rghR1gene, a modified rghR2 gene, a modified yvzC gene and a modified Bli3644 gene, wherein the modified cell produces an increased amount of a protein of interest relative to the parental cell when cultivated under the same conditions.

2. The modified cell of claim 1, wherein the modified rghR1 gene comprises a genetic modification which mutates, disrupts, partially deletes, or completely deletes the encoded RghR1 protein.

3. The modified cell of claim 1, wherein the modified rghR1 gene comprises a genetic modification which mutates, disrupts, partially deletes, or completely deletes a 5-UTR sequence and/or a 3′-UTR sequence of the rghR1 gene, wherein the modified rghR1 gene does not express the encoded RghR1 protein.

4. The modified cell of claim 1, wherein the modified rghR2 gene comprises a genetic modification which mutates, disrupts, partially deletes, or completely deletes the encoded RghR2 protein.

5. The modified cell of claim 1, wherein the modified rghR2 gene comprises a genetic modification which mutates, disrupts, partially deletes, or completely deletes a 5-UTR sequence and/or a 3′-UTR of the rghR2 gene, wherein the modified rghR2 gene does not express the encoded RghR2 protein.

6. The modified cell of claim 1, wherein the modified rghR1 gene and modified rghR2 gene comprise a genetic modification which mutates. disrupts, partially deletes, or completely deletes the encoded RghR1 protein and RghR2, respectively.

7. The modified cell of claim 1, wherein the modified rghR1gene and modified rghR2 gene comprise comprises a genetic modification which mutates, disrupts, partially deletes, or completely deletes a 5′-UTR sequence and/or a 3′-UTR of the rghR1 gene and a 5′-UTR sequence and/or a 3′-UTR of the rghR2 gene, wherein the modified rghR1 gene does not express the encoded RghR1 protein and the modified rghR2 gene does not express the encoded RghR2 protein, respectively.

8. The modified cell of claim 1, wherein the modified rghR1, rghR2, yvzC and Bli3644 genes comprise a genetic modification which mutates, disrupts, partially deletes, or completely deletes the encoded RghR1 protein, the encoded RghR2 protein, the encoded YvzC protein and the encoded Bli3644 protein, respectively.

9. The modified cell of claim 1, wherein the modified rghR1, rghR2, yvzC and Bli3644 genes comprise a genetic modification which mutates, disrupts, partially deletes. or completely deletes a 5′-UTR sequence and/or a 3′-UTR of the rghR1 gene, a 5′-UTR sequence and/or a 3′-UTR of the rghR2 gene, a 5′-UTR sequence and/or a 3′-UTR of the yvzC gene, and a 5′-UTR sequence and/or a 3′-UTR of the Bli3644 gene, wherein the modified rghR1 gene does not express the encoded RghR1 protein, the modified rghR2 gene does not express the encoded RghR2 protein, the modified yvzC gene does not express the encoded yvzC protein and the modified Bli3644 gene does not express the encoded Bli3644 protein, respectively.

10. (canceled)

11. The modified cell of claim 1, comprising one or more expression cassettes encoding a protein of interest.

12. The modified cell of claim 11, wherein the one or more expressions cassettes encode an amylase protein.

13. A modified Bacillus licheniformis cell derived from a parental B. licheniformis cell comprising a native rghR2 gene. wherein the modified cell comprises at least one genetic modification which mutates, disrupts, partially deletes, or completely deletes the rghR2 gene, wherein the modified cell produces a reduced amount of red pigment relative to the parental cell when cultivated under the same conditions.

14. The modified cell of claim 13, wherein the red pigment is further defined as pulcherriminic acid.

15. The modified cell of claim 13, comprising one or more expression cassettes encoding a protein of interest.

16. The modified cell of claim 15, wherein the one or more expressions cassettes encode an amylase protein.

17. The modified cell of claim 13, wherein the modified cell produces an increased amount of a protein of interest relative to the parental cell.

18. A method for producing an increased amount of a protein of interest in a modified Bacillus licheniformis cell comprising:
(a) obtaining a parental B. licheniformis cell and genetically modifying at least one gene of the rghR locus selected from the group consisting of: (i) a rghR1 gene. (ii) a rghR2 gene, (iii) a yvzC gene and (iv) a Bli3644 gene, or a combination thereof, and
(b) fermenting the modified cell under suitable conditions for the production of a protein of interest,
wherein the modified cell produces an increased amount of a protein of interest relative to the parental cell when cultivated under the same conditions.

19-22. (canceled)

23. The method of claim 18, wherein the cell comprises one or more expression cassettes encoding a protein of interest.

24-25. (canceled)

26. A method for producing a protein of interest in modified Bacillus licheniformis cell, wherein the modified cell produces a reduced amount of red pigment during fermentation, the method comprising:
(a) obtaining a parental B. licheniformis cell and genetically modifying the rghR2 gene of the rghR locus, and
(b) fermenting the modified cell under suitable conditions for the production of a protein of interest,
wherein the modified cell produces a reduced red pigment relative to the parental cell when cultivated under the same conditions.

27. (canceled)

28. The method of claim 26, wherein the cell comprises one or more expression cassettes encoding a protein of interest.

29. (canceled)

30. The method of claim 28, wherein the modified cell produces an increased amount of the protein of interest, relative to the parental cell when cultivated under the same conditions.
